(19) United States
(12) Patent Application Publication
(10) Pub. No.: US 2023/0096781 A1
(43) Pub. Date: Mar. 30, 2023

(54) METHODS OF CONTROLLING BODYWEIGHT BY MODULATING PHOSPHATIDYLINOSITOL 5-PHOSPHATE 4-KINASE BETA ACTIVITY

(71) Applicant: University of Cincinnati, Cincinnati, OH (US)

(72) Inventor: Atsuo Sasaki, Cincinnati, OH (US)

(73) Assignee: University of Cincinnati, Cincinnati, OH (US)

(21) Appl. No.: 17/909,187

(22) PCT Filed: Mar. 2, 2021
(86) PCT No.: PCT/US2021/020405

§ 371 Date: Sep. 2, 2022

Related U.S. Application Data
(60) Provisional application No. 62/984,026, filed on Mar. 2, 2020.

(51) Int. Cl. A01K 67/027; A61K 31/365; A61K 31/7105; A61P 21/00; A61P 3/04; C12N 9/12

(52) U.S. Cl. CPC A01K 67/0278; A61K 31/365; A61K 31/7105; A61P 21/00; A61P 3/04; C12N 9/1205; C12Y 101/01205; C12Y 207/01149; C12Y 603/05002; A01K 2217/072; A01K 2227/105; A01K 2267/0306

(57) ABSTRACT

A method for treating a metabolic disorder associated with abnormal bodyweight in a subject is provided, the method including administering to the subject an effective amount of a compound that modulates phosphatidylinositol 5-phosphate 4-kinase beta (PI5P4Kβ) activity, wherein a PI5P4Kβ inhibitor is administered when the subject suffers from a metabolic disorder associated with an underweight bodyweight; and wherein a PI5P4Kβ agonist is administered when the subject suffers from a metabolic disorder associated with an overweight or obese bodyweight. Also provided herein are methods of increasing meat quality and/or yield in livestock or domesticated poultry by administering to an animal an effective amount of a PI5P4Kβ inhibitor, and genetically engineered animals having a substitution in PI5P4Kβ that reduces its GTP-sensing activity.

### CROSS-REFERENCE TO RELATED APPLICATIONS

This application claims priority to U.S. Provisional Application Ser. No. 62/984,026, filed Mar. 2, 2020, the entire contents of which are incorporated herein by reference.

### STATEMENT REGARDING FEDERALLY SPONSORED RESEARCH OR DEVELOPMENT

This invention was made with Government support under 5R01NS089815-05 awarded by National Institutes of Health. The Government has certain rights in the invention.

### TECHNICAL FIELD

The present disclosure relates to the field of bodyweight management. More specifically, the disclosure relates to modulation of the GTP energy sensor phosphatidylinositol 5-phosphate 4-kinase beta (PI5P4Kβ) for applications in the therapeutic treatment of cachexia, obesity, and in improvement of meat quality and yield in animal husbandry.

### SEQUENCE LISTING

Applicant incorporates by reference a CRF sequence listing submitted herewith having file name Sequence_Listing_10738_847.txt, created on Feb. 28, 2021. The nucleic acid and/or amino acid sequences listed in the accompanying sequence listing are shown using standard abbreviations as defined in 37 C.F.R. 1.822.

### BACKGROUND

Cachexia is an involuntary wasting disorder associated with severe chronic illness or burn injury. Patients with advanced cachexia are characterized by anorexia, early satiety, severe weight loss, muscle wasting, loss of body fat, weakness, anemia, and edema. Individuals suffering from serious diseases such as cancer, AIDS, heart failure, kidney disease, and the like may suffer with cachexia as the body fights the disease. It is thought that cachexia results as the individual loses appetite and the body begins to burn calories more quickly. The individual thus loses weight, as the body shifts energy to the brain and begins to break down muscle tissue and fat stores. Cachexia weakens the body further, rendering the individual more susceptible to secondary infections.

Cachexia occurs in approximately 50% of all cancer patients and may be the direct result of the disease or a consequence of its treatment. It is considered that cachexia can interfere with radio- or chemotherapy and that its management can improve outcomes and provide a sense of well-being for patients and their families.

In relation to the approval of novel therapeutics for cachexia, regulatory authorities suggest it is important not only to show efficacy for improved nutritional status such as lean body mass (LBM) but also functional status such as performance status. Poor physical function in cachexia may relate to many factors, including loss of body mass, reduced substrate supply (food), reduced vitality, increased mortality, and increased fatigue and depression.

While progestins, corticosteroids, metoclopramide, cannabinoids, thalidomide, melatonin, clenbuterol, anabolic steroids, omega 3 fatty acids and NSAIDs are used as the treatments for cachexia, the therapeutic benefits thereof have been limited and a need exists for improved therapies to reverse the effects of cachexia and assist patients in regaining weight.

### SUMMARY

The present disclosure demonstrates that the GTP-sensing activity of PI5P4Kβ is important for bodyweight control. The molecular mechanism of GTP-recognition is identified, revealing the critical motif for GTP sensing. The discoveries of the GTP-sensing activity in bodyweight control along with the discovery of the “tunability” of GTP-dependent activity by administering PI5P4Kβ inhibitors or agonists have applications in weight management for underweight and overweight individuals, as well as in animal husbandry.

In one embodiment, a method for treating a metabolic disorder associated with abnormal bodyweight in a subject in need thereof is provided, the method comprising administering to the subject an effective amount of a compound that modulates phosphatidylinositol 5-phosphate 4-kinase beta (PI5P4Kβ) kinase activity, wherein a PI5P4Kβ inhibitor is administered when the subject suffers from a metabolic disorder associated with an underweight bodyweight; and wherein a PI5P4Kβ agonist is administered when the subject suffers from a metabolic disorder associated with an overweight or obese bodyweight.

In another embodiment, a method for treating cachexia in a subject in need thereof is provided, the method comprising administering to the subject an effective amount of a PI5P4Kβ inhibitor.

In another embodiment, a method for method for reducing excess bodyweight in a subject in need thereof is provided, the method comprising administering to the subject an effective amount of a PI5P4Kβ agonist.

In another embodiment, a method for increasing the bodyweight of an animal is provided, the method comprising administering to the animal an effective amount of a phosphatidylinositol 5-phosphate 4-kinase beta (PI5P4Kβ) inhibitor.

In another embodiment, a knock-in animal comprising a F205L substitution in PI5P4Kβ is provided, having utility in research and animal husbandry. In a specific embodiment, a knock-in animal whose genome encodes a mutant PI5P4Kβ kinase is provided, wherein said mutant PI5P4Kβ kinase comprises at least a F205L substitution, wherein the knock-in animal has decreased GTP-sensing activity of the PI5P4Kβ kinase compared to wildtype animals lacking the substitution.

These and other objects, features, embodiments, and advantages will become apparent to those of ordinary skill in the art from a reading of the following detailed description and the appended claims.

### BRIEF DESCRIPTION OF THE DRAWINGS

FIG. 1 demonstrates the bodyweight difference in F205L knock-in genetically engineered mice in which endogenous Pip4k2b gene (coding PI5P4Kβ) is introduced a mutation (Phe-205 with Leucine) to decrease GTP-sensing activity compared to heterozygous mice. (A) is in image of heterozygous Pip4k2b^{WT/F25L} mice, which are viable and appear normal in appearance and activity. (B) is an image of homozygous Pip4k2b^{F205L/F205L} mice, which are viable and appear normal in appearance and activity. However, homozygous Pip4k2b^{F205L/F205L} demonstrate increased bodyweight and some (e.g., left mouse) show remarkable increase. (C) is a graph comparing bodyweight analysis of Pip4k2b^{WT/WT} and Pip4k2b^{F205L/F205L} mice.

FIG. 2 demonstrates the GTP-sensing activity of PI5P4Kβ is important for whole-body glucose metabolism and liver architecture in male mice. (A) is an image showing histological abnormalities of liver from Pip4k2b^{F205L/F205L} mice. (B) is a graph comparing body composition (left) and energy intake and expenditure (right) of WT and Pip4k2b^{F205L/F205L} mice. (C) shows results of a respiratory exchange rate (RER) test in WT and Pip4k2b^{F205L/F205L} mice (n=10). (D) shows graphs comparing basal glucose level under fed and fasted conditions from WT, Pip4k2b−/− (KO) and Pip4k2b^{F205L/F205L} (FL) mice (n=6). (E) provides graphs showing insulin tolerance under fed condition (left) and blood glucose levels after insulin injection of WT, KO, and FL mice (right) (n=6). (F) are graphs showing glucose tolerance under fed condition (left) and blood glucose levels after insulin injection of WT, KO, and FL mice (right) (n=6).

FIG. 3 demonstrates the GTP-sensing activity of PI5P4Kβ is important for liver function. (A) is a graph comparing glucagon stimulation (left) and change in blood glucose (right) under fed condition of WT, Pip4k2b^{-/-} (KO) and Pip4k2b^{F205L/F205L} (FL) male mice (n=6). (B) shows results of a pyruvate tolerance test under fasting conditions of WT, KO and FL male mice (n=6). (C) is a graph comparing bodyweight after 6 months of high-fat diet, compared to control normal diet during the period (n=5). (D) provides images showing liver sections from WT and Pip4k2b^{F205L/F205L} mice fed a high-fat diet, showing showed severe steatosis in Pip4k2b^{F205L/F205L} mice.

FIG. 4 shows images of lysotracker staining of MEF cells under control conditions (left panels) or serum starvation for 4 h (right panels), which show that primary MEFs from Pip4k2b^{F205L/F205L} mice (bottom panels) have decreased lysosomal acidification compared to MEFs from Pip4k2b^{W/WT} mice (top panels).

FIG. 5 shows images of lysotracker staining of MEFs treated with DMSO, 10 μM mycophenolic acid (MPA), or 20 μM Link17 (PI5P4K inhibitor) in WT-PI5P4Kβ-expressing Pip4K2b^{-/-} cells (top panels) and isogenic Pip4K2b^{-/-} cells. Treatment of Link17 and MPA suppressed lysosomal acidification in serum starved WT-PI5P4Kβ-expressing Pip4K2b^{-/-} cells but not isogenic Pip4K2b^{-/-} cells

FIG. 6 shows the effects of treatment of MEF cells with Link17 and nigericin. (A)-(B) shows treatment with Link17 increased aggregation of mutant Huntington proteins in the WT-PI5P4Kβ/Pip4K2b^{-/-} cells. (C) shows nigericin-dependent vacuolization was suppressed by Link17 in Pip4K2b^{-/-} cells. (D) is a graph showing cytotoxic death by nigericin was suppressed in Pip4K2b^{-/-} cells and F205L-PI5P4Kβ-expressing Pip4K2b−/− cells.

FIG. 7 shows the effects of treatment of MEF cells with Link17. (A) shows Link17-treated cells decreased LC3-II induction in WT-PI5P4Kβ MEFs. (B) shows treatment of Link17 decreased autophagy activity, as assessed by Bafilomycin A1 (BafA1) treatment and monitoring LC3-II accumulation. (C) shows autophagy flux of Pip4k2b^{-/-} cells and F205L-reconstituted cells was decreased compared to that of WT-PI5P4Kfβ cells, under the nutrient starvation (HBSS).

FIG. 8 shows Western blot analysis of liver lysates from WT and Pip4k2b^{F205L/F205L} mice.

FIG. 9 provides data regarding V-ATPase. (A) PIPs binding proteins were precipitated from U87MG lysates and analyzed by silver staining and anti-SNX4 antibody by Western blot. (B) is a schematic of V-ATPase. (C) is a PI5P-binding motif (upper panel) and the corresponding sequence of V-ATPase VIA (bottom panel) are depicted (SEQ ID NO: 1, SEQ ID NO: 2, and SEQ ID NO: 3). (D) illustrates computational analysis of human V-ATPase VO subunit, wherein a putative PI5P-binding pocket is circled (two circles). (E) provides images and graphs showing localization of V-ATPase V0 and V1 subunits to lysosomes, visualized by their antibodies and co-immunostaining for lysosome (TMEM192).

FIG. 10 shows the chemical structures of (A) purine nucleotide triphosphates (PNTs) used in the study, and (B) the ATP- and GTP-binding modes of PI5P4Kβ, wherein dotted lines denote hydrogen bonds.

FIG. 11 shows nucleotide-base binding by kinases and G-proteins. (A) shows typical hydrogen bond interactions between nucleotide bases and proteins for an adenine base in kinases. (B) shows typical hydrogen bond interactions between nucleotide bases and proteins for a guanine base in G-proteins.

FIG. 12 shows PNT hydrolysis activity and binding modes of PI5P4Kβ. (A) The PNT hydrolysis activity of PI5P4Kβ. indicative of the kinase activity of PI5P4Kβ. was assessed by the signal intensity ratios of diphosphorylated/triphosphorylated nucleotides after the reaction. The average values from three experiments are shown with error bars (SD). Highly hydrolyzed nucleotides (>0.1 of the ratios) were shown for GTP, ITP, XTP, 6-thio-GTP, and 2a-ATP. (B), (C), and (D) show interactions of the PI5P4Kβ-ITP, PI5P4Kβ-XTP, and PI5P4Kβ-2a-ATP complexes, respectively. Dotted lines represent hydrogen bonds between the PNTs and PI5P4Kβ.

FIG. 13 shows GTP-, ATP-, and XTP-binding modes and effect of mutations on PI5P4Kβ activity for different PNTs. (A)-(C) show PNT hydrolysis activity of mutant PI5P4Kβ as compared to the WT. The ratios of dephosphorylated/triphosphorylated nucleotides after reaction are shown. The N203A activity is almost negligible. (D) demonstrates the ratios of dephosphorylated/triphosphorylated nucleotides after reaction are shown in color depth to compare PNTs hydrolysis activity and specificity among the WT and mutant PI5P4Kβs.

FIG. 14 shows sequence alignment of PI5P4Kβ and PI4P5K family proteins.

FIG. 15 shows fragmental molecular orbital (FMO) calculation of the PI5P4Kβ-GTP complex. (A) shows the energetic contributions of each residue to PI5P4Kβ-guanine base interaction. (B) shows the energetic contributions of each residue of PI5P4Kβ and GTP to the interaction with water molecules that are bound to the NH₂(2) (top) and O(6) (bottom) positions, respectively, of guanine base moieties.

FIG. 16 shows the interaction of (A) ATP and (B) GTP with the kinase CKII.

FIG. 17 shows inhibition of the ³²P-GTP-dependent kinase activity of PI5P4Kβ by cold PNTs. (A) The kinase reaction was carried out in a total of 50 μl of reaction buffer (50 mM HEPES (pH 7.4), 0.2 mM EGTA, and 10 mM MgCl₂) containing 20 μM of PI(5)P (d-myo-phosphatidylinositol 5-phosphate diC16) that was suspended by sonication. 250 μM of γ-³²P radiolabeled GTP was incubated with 1 μg of recombinant PI5P4Kβ for 10 min at room temperature. 20 μM of PI(5)P was used with 3 μM 1,2-dipalmitoyl-phosphatidylserine as the basal lipid. Phosphoinositides were extracted by a methanol/chloroform (1/1, v/v) mix and subjected to a thin-layer chromatography (TLC) assay using heat-activated 2% oxaloacetate-coated silica gel 60 plates. The solvent was 1-propanol/2 M acetic acid (65/35, v/v). In competition assay, 250 μM of ³²P-labeled GTP was mixed with 3.2-400 μM triphosphorylated nucleotides, and the PI(5)P phosphorylation by GTP was monitored by quantifying the amount of radiolabeled PI(4,5)P2. (B) Relative kinase activities in the presence of each PNT against those without them (i.e., only ³²P-GTP condition) are shown.

The details of embodiments of the presently-disclosed subject matter are set forth in this document. Modifications to embodiments described in this document, and other embodiments, will be evident to those of ordinary skill in the art after a study of the information provided in this document.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an” and “the” include plural references unless the content clearly dictates otherwise.

As used herein, the term “subject” refers to any subject having a GTP-sensing PI5P4Kβ kinase gene susceptible to modulation. In embodiments, the subject is a mammalian subject, including humans, non-human primates, pigs, dogs, rats, mice, and the like. In a specific embodiment, the subject is a human patient. In another embodiment, the subject is an animal, such as a livestock animal or domesticated poultry animal. In another specific embodiment, the animal is selected from cattle, sheep, goats, pigs, rabbits, chickens, ducks, geese, turkeys, fish, and the like.

An exemplary amino acid sequence for human PI5P4Kβis set forth as UniProtKB P78356:

(SEQ ID NO: 23)
MSSNCTSTTAVAVAPLSASKTKTKKKHFVCQKVKLFRASEPILSVLMWG
VNHTINELSNVPVPVMLMPDDFKAYSKIKVDNHLFNKENLPSRFKFKEY
CPMVFRNLRERFGIDDQDYQNSVTRSAPINSDSQGRCGTRFLTTYDRRF
VIKTVSSEDVAEMHNILKKYHQFIVECHGNTLLPQFLGMYRLTVDGVET
YMVVTRNVFSHRLTVHRKYDLKGSTVAREASDKEKAKDLPTFKDNDFLN
EGQKLHVGEESKKNFLEKLKRDVEFLAQLKIMDYSLLVGIHDVDRAEQE
EMEVEERAEDEECENDGVGGNLLCSYGTPPDSPGNLLSFPRFFGPGEFD
PSVDVYAMKSHESSPKKEVYFMAIIDILTPYDTKKKAAHAAKTVKHGAG
AEISTVNPEQYSKRFNEFMSNILT.

The term “effective amount” refers to an amount sufficient to achieve beneficial or desired results. An effective amount can be administered in one or more administrations, applications or dosages. The effective amount of the PI5P4Kβ inhibitors or agonists for use in the methods herein will vary with the metabolic disorder being treated, the age and physical condition of the subject to be treated, the severity of the condition, the duration of the treatment, the nature of concurrent therapy, the particular therapeutic agents being employed, and like factors within the knowledge and expertise of the attending physician.

As used herein, the term “metabolic disorder” refers to a disease or condition associated with an abnormal bodyweight. In some embodiments, a metabolic disorder is associated with the condition of being underweight, for example, due to cachexia associated with severe illness, trauma, surgery, burn injury, etc. In some embodiments, a metabolic disorder is associated with the condition of being overweight or obese. Such metabolic disorders include, but are not limited to, obesity, type II diabetes, non-alcoholic fatty liver disease, chronic heart failure, kidney failure, and the like.

It is generally understood that a normal bodyweight for humans is characterized by a body mass index (BMI) of 18.5-24.9 kg/m². The term “underweight,” as used herein, refers to a subject whose bodyweight corresponds to a BMI less than 18.5 kg/m². The term “overweight,” as used herein, refers to a subject whose bodyweight corresponds to a BMI is 25.0 or greater. The term “obese,” as used herein, refers to a subject whose bodyweight corresponds to a BMI of 30.0 kg/m² or greater. BMI is calculated by dividing a subject's mass in kg by the subject's height in meters squared: BMI=mass (kg)/height² (m).

“Livestock” as used herein refers to domesticated animals raised for meat consumption, including, but not limited to, cattle, sheep, goats, pigs, rabbits, horses, fish, frogs, lobster, crab, squid, locust, spiders, worms, and the like.

“Domesticated poultry” refers to fowl raised for meat consumption, including but not limited to chickens, ducks, geese, turkeys, and the like.

“Increased meat quality” refers to an improved tenderness, flavor, juiciness, or color compared to meat obtained from untreated animals. In embodiments, animals raised for consumption and treated with PI5P4Kβ inhibitors as disclosed herein have increased meat quality compared to untreated animals. In embodiments, genetically engineered animals having a mutation that decreases the GTP-sensing activity of PI5P4Kβ have increased meat quality compared to wildtype counterparts. In embodiments, increased meat quality refers to beef having a USDA beef grade of “choice” or “prime.” In other embodiments, increased meat quality refers to beef having a Japanese Meat Grading Association grade of 4 or 5.

“Increased meat yield” refers to an increased bodyweight corresponding to muscle, connective tissues, organs, and/or fat compared to bodyweight of untreated animals. In embodiments, animals raised for consumption and treated with PI5P4Kβ inhibitors as disclosed herein have an increased meat yield compared to untreated animals. In embodiments, genetically engineered animals having a mutation that decreases the GTP-sensing activity of PI5P4Kβ have an increased meat yield compared to wildtype counterparts. In specific embodiments, increased meat yield refers to a meat yield that is increased by a statistically significant amount compared to meat yield from an untreated animal or a wildtype animal lacking a mutation to increase meat yield as disclosed herein.

Kinases are essential for a variety of cellular processes, including signal transduction, transcription, and metabolism. There is extraordinary diversity in their structure, substrate specificity, and participating pathways. Protein kinases, which represent the largest superfamily consisting of over 500 different distinct genes in the human genome, share a conserved catalytic domain and structural motif that serves for ATP recognition and catalysis. On the other hand, phosphoinositide kinases and inositol phosphate kinases (IP-kinase, including inositol kinases) form distinct families that target the inositol moieties of substrates. Although the families of phosphoinositide and IP-kinases have distinct folds from protein kinases, all these kinases use ATP as the physiological phosphate donor.

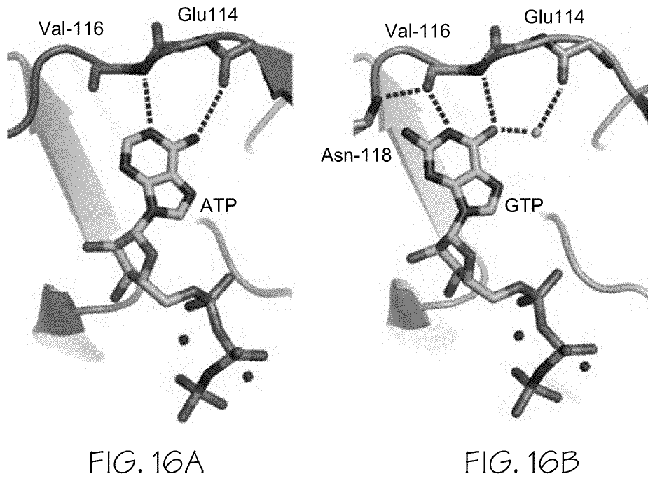

The preference for ATP has been experimentally defined for more than 200 kinases, most of which have a more than 3-fold preference for ATP over GTP based on their affinity values. While GTP is the second-most abundant triphosphorylated nucleotide in cells (0.1-0.5 mM), the affinity difference coupled the higher physiological concentration of ATP (1-5 mM) result in the occupation of kinase catalytic centers by ATP under most cellular physiological conditions. The guanine base cannot interact in the same way as the adenine base in the nucleotide binding pocket, due to the distinct hydrogen donors and acceptors at the 1st and 6th positions of guanine and adenine. There are only a few examples of kinases, such as casein kinase II (CKII), that react equally well with GTP and ATP (FIG. 16).

The greater frequency of ATP-preferring kinases has given rise to the belief that kinase function depends on ATP. Given this prevailing notion, the strong GTP-preference of phosphatidylinositol 5-phosphate 4-kinase β (PI5P4Kβ) was a surprising discovery. PI5P4K, also called Type II PIPK, is a member of the phosphoinositide kinase superfamily and converts the second lipid messenger phosphatidylinositol 5-phosphate (PI(5)P) to phosphatidylinositol 4,5-diphosphate (PI(4,5)P2). Despite the higher intracellular concentration of ATP, PI5P4Kβ exhibits a strong preference for GTP and a K_{M} value (K_{M} for GTP ˜88 μM) that is well within the physiological variation of GTP concentration. Importantly, a structure-based reverse genetic analysis demonstrated that PI5P4Kβ acts as an intracellular GTP sensor. Interestingly, an evolutionarily cognate phosphoinositide-kinase, PI4P5K/Type I PIPK, utilizes ATP for its reaction (Kazutaka Sumita, et al., The Lipid Kinase PI5P4Kβ is an Intracellular GTP Sensor for Metabolism and Tumorigenesis, Molecular Cell 61: 187-98 (2016)). A recent report suggests that the divergence of PI5P4K from the PI4P5K family likely occurred at the ancestral lineage of Choanoflagellates and Filasterea. The PI5P4K genes are found in a variety of organisms belonging to the Holozoa Glade of eukaryotes; however, these genes are not found in the deeper-branching eukaryotic lineages, or in either plants or fungi. Therefore, PI5P4Kβ represents an intriguing example of evolutionary switching of nucleotide preference from ATP to GTP. Considering the high sequence identity between the PI5P4Kβ and PI4P5K subfamilies (>60%), analysis of the amino acid substitutions in the catalytic pocket serve to uncover the structural requirement that allowed PI5P4Kβ to functionally evolve to an intra-cellular GTP-sensor during the development and homeostasis of multicellular animals.

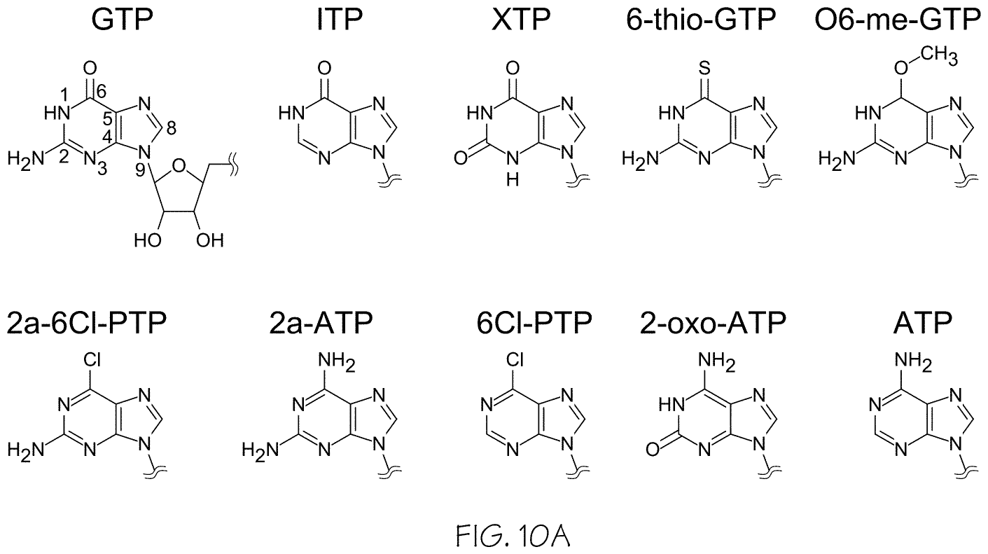

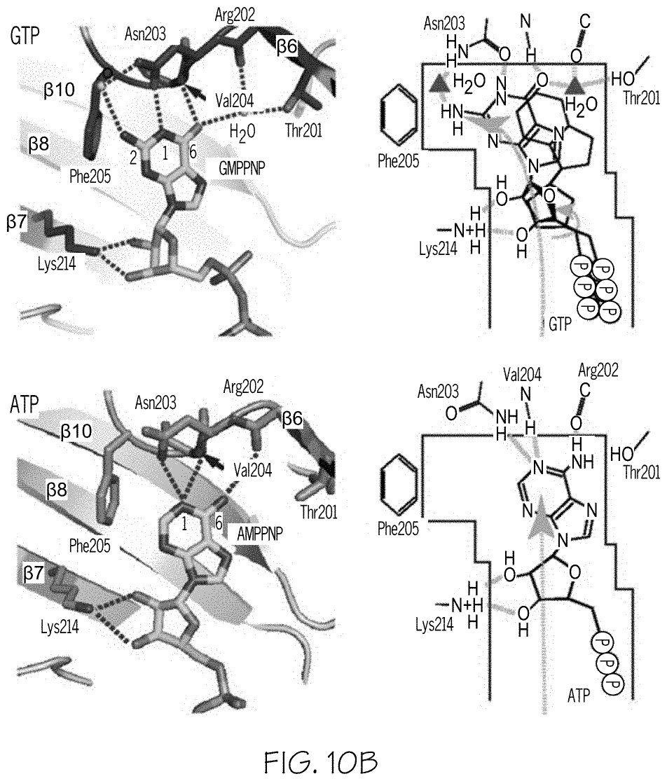

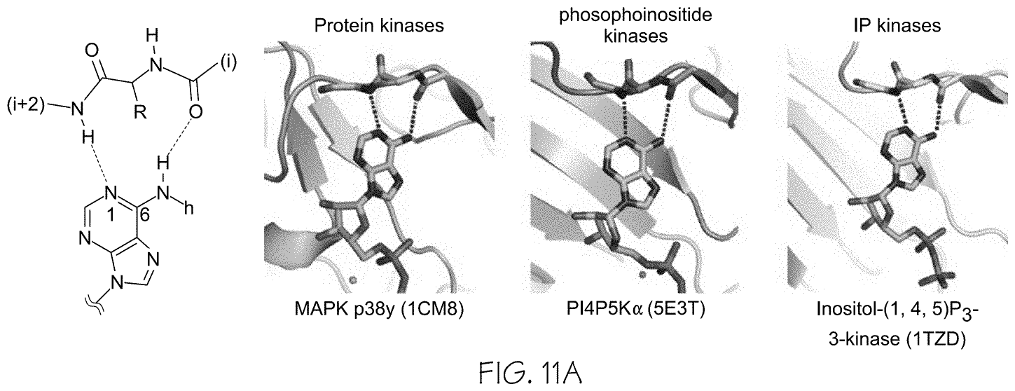

The present disclosure biochemically and structurally characterizes the nucleotide preference of PI5P4Kβ by a systematic utilization of 10 different purine nucleotide triphosphates (PNTs) (FIG. 10(A)) and introduction of amino-acid substitutions to the nucleotide-binding pocket. These analyses reveal a trade-off relationship between the GTP-dependent activity and nucleotide specificity of PI5P4Kβ. The results assist in understanding how PI5P4Kβ acquired a GTP preference to function as an intracellular GTP sensor.

The present inventors have discovered that the short nucleotide base-recognition motif, TRNVF (SEQ ID NO: 4), is responsible for the GTP binding activity of PI5P4Kβ. Further, the data presented herein show that the GTP-sensing activity of PI5P4Kβ is implicated in bodyweight control and can be modulated up or down by agonists or inhibitors, respectively, to effect a change in bodyweight.

In one embodiment, a method for treating a metabolic disorder associated with abnormal bodyweight in a subject in need thereof is provided, the method comprising administering to the subject an effective amount of a compound that modulates phosphatidylinositol 5-phosphate 4-kinase beta (PI5P4Kβ) kinase activity, wherein a PI5P4Kβ inhibitor is administered when the subject suffers from a metabolic disorder associated with an underweight bodyweight; and wherein a PI5P4Kβ agonist is administered when the subject suffers from a metabolic disorder associated with an overweight or obese bodyweight.

In embodiments, the metabolic disorder is selected from the group consisting of cachexia, obesity, type II diabetes, and nonalcoholic fatty liver disease. In a specific embodiment, the metabolic disorder is cachexia. In another specific embodiment, the metabolic disorder is obesity.

In embodiments, the metabolic disorder is cachexia and the compound that modulates PI5P4Kβ kinase activity is a PI5P4Kβ inhibitor.

In embodiments, PI5P4Kβ inhibitors are compounds that bind at least in part to the GTP-binding pocket of PI5P4Kβ and down-regulate the kinase activity of PI5P4Kβ. For example, in embodiments, the PI5P4Kβ inhibitor binds to the TRNVF (SEQ ID NO: 4) motif of the kinase, interfering with the GTP-sensing capacity of the kinase and thereby down-regulating its activity.

The structure-activity relationship of PI5P4Kβ inhibitors is described by Manz, et al., Structure-Activity Relationship Study of Covalent Pan-phosphatidylinositol 5-Phosphate 4-Kinase Inhibitors, ACS Med Chem Lett. 11(3): 346-52 (2019).

Various PI5P4Kβ inhibitors are known in the art and suitable for use in the presently disclosed methods. PI5P4Kβ inhibitors include, but are not limited to, 6-thioguanine, I-OMe tyrphostin AG 538, A131, SAR088, NIH-12848, NCT-504, THZ-P1-2, inosine monophosphate dehydrogenase (IMPDH) inhibitors, guanosine monophosphate synthetase (GMPS) inhibitors, and combinations thereof.

In a specific embodiment, the PI5P4Kβ inhibitor is an IMPDH inhibitor. In a more specific embodiment, the IMPDH inhibitor is selected from the group consisting of mycophenolic acid (MPA), mycophenylate sodium, mycophenylate mofetil, tiazofurin, ribavirin, VX-944, FF-10501, benzamide riboside, mizorbine, 5-ethynyl-1-beta-D-ribofuranosylimidazole-4-carboxamide (EICAR), selenazofurin, thiophenfurin, myricetin, gnidilatimonoein, sappanone A, sanglifehrin, and combinations thereof. In a very specific embodiment, the PI5P4Kβ inhibitor is selected from MPA, mycopheylate sodium, mycophenylate mofetil, and combinations thereof. Suitable IMPDH inhibitors are found, for example, in Naffouje, et al., Anti-Tumor Potential of IMP Dehydrogenase Inhibitors: A Century-Long Story, Cancers 11(9): 1346 (2019).

In another specific embodiment, the PI5P4Kβ inhibitor is a GMPS inhibitor. In a more specific embodiment, the GMPS inhibitor is selected from the group consisting of acivicin, angustmycin A, decoyinine, oxanosine, and combinations thereof. Suitable GMPS inhibitors are found, for example, in Itoh, et al., Induction by the Guanosine Analogue Oxanosine of Reversion toward the Normal Phenotype of K-ras-transformed Rat Kidney Cells, Cancer Research 49(4): 1989.

In another embodiment, the metabolic disorder is selected from the group consisting of obesity, type II diabetes, and non-alcoholic fatty liver disease and the compound is a PI5P4Kβ agonist.

PI5P4Kβ agonists are compounds that increase the concentration of GTP in a cell of the subject. Various PI5P4Kβ agonists are known in the art. Suitable PI5P4Kβ agonists include, but are not limited to, hypoxanthine, guanine, guanosine, inosine, guanosine monophosphate (GMP), guanosine diphosphate (GDP), guanosine triphosphate (GTP), inosine triphosphate (ITP), xanthosine triphosphate (XTP), and combinations thereof.

In another embodiment, the method of further comprises administration of an effective amount of a second active agent selected from the group consisting of glucagon, leptin, adrenalin, incretin, nicotinamide mononucleotide, vitamin B group, caffeine, orlistat/vyfat//tetrahydrolipstatin, non-steroidal anti-inflammatory drugs, (NSAIDs), beta-adrenergic receptor antagonists, catabolic steroids, and combinations thereof.

In another embodiment, a method for treating cachexia in a subject in need thereof is provided, the method comprising administering to the subject an effective amount of a PI5P4Kβ inhibitor. In a specific embodiment, the subject is a mammal. In a more specific embodiment, the subject is a human.

In embodiments, the PI5P4Kβ inhibitor is selected from the group consisting of 6-thioguanine, I-OMe tyrphostin AG 538, A131, SAR088, NIH-12848, NCT-504, THZ-P1-2, inosine monophosphate dehydrogenase (IMPDH) inhibitors, guanosine monophosphate synthetase (GMPS) inhibitors, and combinations thereof.

Cachexia may result from severe illness, trauma, surgery, or burn injury in the subject. In embodiments, severe illnesses include, but are not limited to, cancer, AIDS, HIV infection, chronic heart failure, kidney disease, and the like.

When the cachexia is a result of concomitant cancer in the subject, the methods set forth herein optionally further comprise administration of one or more anti-cancer therapeutics to the subject.

In other embodiments, treatment of cachexia in the individual may further comprise administration to the subject of an effective amount of a second active agent selected from the group consisting of propranolol, beta-adrenergic receptor blockers, recombinant human growth hormone, progestin, corticosteroids, metoclopramide, cannabinoids, thalidomide, melatonin, clenbuterol, anabolic steroids, omega 3 fatty acids, non-steroidal anti-inflammatory drugs, (NSAIDs), and combinations thereof.

Administration with additional active agents includes substantially concurrent administration or sequential administration.

In another embodiment, a method for method for reducing excess bodyweight in a subject in need thereof is provided, the method comprising administering to the subject an effective amount of a PI5P4Kβ agonist. In embodiments, the subject is overweight, and may have a BMI of 25.0 or greater. In embodiments, the subject is obese, and may have a BMI of 30.0 or greater.

In embodiments, the PI5P4Kβ agonist is selected from the group consisting of hypoxanthine, guanine, guanosine, inosine, guanosine monophosphate (GMP), guanosine diphosphate (GDP), guanosine triphosphate (GTP), inosine triphosphate (ITP), xanthosine triphosphate (XTP), and combinations thereof.

In another embodiment, the method further comprises administration of an effective amount of a second active agent selected from the group consisting of glucagon, leptin, adrenalin, incretin, nicotinamide mononucleotide, vitamin B group, caffeine, orlistat/vyfat//tetrahydrolipstatin, non-steroidal anti-inflammatory drugs, (NSAIDs), beta-adrenergic receptor antagonists, catabolic steroids, and combinations thereof.

In other embodiments, modulation of the GTP-sensing faculty of PI5P4Kβ has application in animal husbandry. Globally, regions of the world continue to face problems associated with food shortages. The “tunability” of PI5P4Kβ kinase function may be exploited to enhance the bodyweight of livestock or domestic poultry in order to increase meat yield and/or increase/improve meat quality.

In embodiments, a method for increasing the bodyweight of an animal is provided, the method comprising administering to the animal an effective amount of a phosphatidylinositol 5-phosphate 4-kinase beta (PI5P4Kβ) inhibitor. In embodiments, the animal is a livestock animal or a domesticated poultry animal. In embodiments, the animal is selected from the group consisting of cattle, sheep, goats, pigs, rabbits, chickens, ducks, geese, turkeys, horses, fish, frogs, lobster, crap, squid, locust, spiders, worms, and the like.

In embodiments, the PI5P4Kβ inhibitor is selected from the group consisting of 6-thioguanine, I-OMe tyrphostin AG 538, A131, SAR088, NIH-12848, NCT-504, THZ-P1-2, IMPDH inhibitors, GMPS inhibitors, and combinations thereof.

In still another embodiment, an animal engineered to have decreased GTP-sensing activity of PI5P4Kβ is provided. Optionally, the animal is genetically engineered to include a mutation that decreases the GTP-sensing activity of PI5P4Kβ. For example, in embodiments, an animal having a Phe205Leu (F205L) mutation in PI5P4Kβ protein is provided. In embodiments, the genetically engineered animal is selected from the group consisting of rodents (mice, rats, etc.), cattle, sheep, goats, pigs, rabbits, chickens, ducks, geese, turkeys, horses, fish, frogs, lobster, crap, squid, locust, spiders, worms, and the like. Animals engineered as disclosed herein have utility in research and animal husbandry.

In another embodiment, a knock-in animal comprising a F205L substitution in PI5P4Kβ is provided, having utility in research and animal husbandry. In a specific embodiment, a knock-in animal is provided, whose genome encodes a mutant PI5P4Kβ kinase, wherein said mutant PI5P4Kβ kinase comprises at least one F205L or analogous substitution, wherein the knock-in animal has decreased GTP-sensing activity of the PI5P4Kβ kinase. In a specific embodiment, the animal is a mouse.

Such animals may be genetically engineered according to methods known in the art. In a specific embodiment, the animal is a knock-in animal and the F205L mutation is generated by the CRISPR/Cas9 method for gene editing (CRISPR Therapeutics, Cambridge, Mass.). In a specific embodiment, the mouse is a C57BL/6 mouse having an introduced F205L mutation. In a very specific embodiment, the mouse is a C57BL/6J mouse.

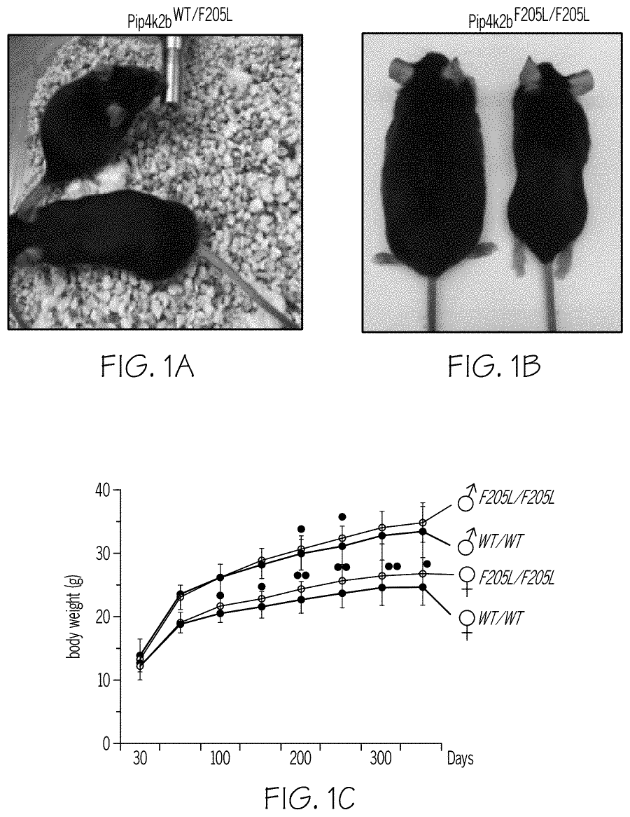

Animals genetically engineered to have decreased GTP-sensing activity of PI5P4Kβ tend to develop increased bodyweights compared to non-mutated control animals (FIG. 1). Introduction of the F205L (or an analogous mutation to the GTP binding pocket of PI5P4Kβ) into an animal by gene editing permits the production of animal strains that tend to have increased bodyweights compared to wildtype animals. Such genetically engineered animals are useful in animal husbandry and food production, as the meat obtained from such animals has enhanced quality and yield compared to wildtype animals.

Embodiments can be described with reference to the following numbered clauses, with preferred features laid out in dependent clauses.
1. A method for treating a metabolic disorder associated with abnormal bodyweight in a subject in need thereof, the method comprising administering to the subject an effective amount of a compound that modulates phosphatidylinositol 5-phosphate 4-kinase beta (PI5P4Kβ) kinase activity,

wherein a PI5P4Kβ inhibitor is administered when the subject suffers from a metabolic disorder associated with an underweight bodyweight; and

wherein a PI5P4Kβ agonist is administered when the subject suffers from a metabolic disorder associated with an overweight or obese bodyweight.
2. The method according to clause 1, wherein the metabolic disorder is selected from the group consisting of cachexia, obesity, type II diabetes, and non-alcoholic fatty liver disease.
3. The method according to any of the preceding clauses, wherein the metabolic disorder is cachexia and the compound is a PI5P4Kβ inhibitor.
4. The method according to any of the preceding clauses, wherein the PI5P4Kβ inhibitor is selected from the group consisting of 6-thioguanine, I-OMe tyrphostin AG 538, A131, SAR088, NIH-12848, NCT-504, THZ-P1-2, and combinations thereof.
5. The method of according to any of the preceding clauses, wherein the PI5P4Kβ inhibitor comprises an inosine monophosphate dehydrogenase (IMPDH) inhibitor.
6. The method according to clause 5, wherein the IMPDH inhibitor is selected from the group consisting of mycophenolic acid (MPA), mycophenylate sodium, mycophenylate mofetil, tiazofurin, ribavirin, VX-944, FF-10501, benzamide riboside, mizorbine, 5-ethynyl-1-beta-D-ribofuranosylimidazole-4-carboxamide (EICAR), selenazofurin, thiophenfurin, myricetin, gnidilatimonoein, sappanone A, sanglifehrin, and combinations thereof.
7. The method according to clause 6, wherein the IMPDH inhibitor is MPA, mycophenylate sodium, mycophenylate mofetil, or combinations thereof.
8. The method according to clause 3, wherein the PI5P4Kβ inhibitor comprises a guanosine monophosphate synthetase (GMPS) inhibitor.
9. The method according to clause 8, wherein the GMPS inhibitor is selected from the group consisting of acivicin, angustmycin A, decoyinine, oxanosine, and combinations thereof.
10. The method according to clause 1, wherein the metabolic disorder is selected from the group consisting of obesity, type II diabetes, and non-alcoholic fatty liver disease and the compound is a PI5P4Kβ agonist.
11. The method according to clause 10, wherein the PI5P4Kβ agonist is selected from the group consisting of hypoxanthine, guanine, guanosine, inosine, guanosine monophosphate (GMP), guanosine diphosphate (GDP), guanosine triphosphate (GTP), inosine triphosphate (ITP), xanthosine triphosphate (XTP), and combinations thereof
12. A method for treating cachexia in a subject in need thereof, the method comprising administering to the subject an effective amount of a phosphatidylinositol 5-phosphate 4-kinase beta (PI5P4Kβ) inhibitor.
13. The method according to clause 12, wherein the subject is a mammal.
14. The method according to any of clauses 12-13, wherein the subject is a human.
15. The method according to any of clauses 12-14, wherein the PI5P4Kβ inhibitor is selected from the group consisting of 6-thioguanine, I-OMe tyrphostin AG 538, A131, SAR088, NIH-12848, NCT-504, THZ-P1-2, inosine monophosphate dehydrogenase (IMPDH) inhibitors, guanosine monophosphate synthetase (GMPS) inhibitors, and combinations thereof.
16. The method according to any of clauses 12-15, wherein the PI5P4Kβ inhibitor is an IMPDH inhibitor selected from the group consisting of mycophenolic acid (MPA), mycophenylate sodium, mycophenylate mofetil, tiazofurin, ribavirin, VX-944, FF-10501, benzamide riboside, mizorbine, 5-ethynyl-1-beta-D-ribofuranosylimidazole-4-carboxamide (EICAR), selenazofurin, thiophenfurin, myricetin, gnidilatimonoein, sappanone A, sanglifehrin, oxanosine, and combinations thereof.
17. The method according to clause 15, wherein the PI5P4Kβ inhibitor is a GMPS inhibitor selected from the group consisting of acivicin, angustmycin A, decoyinine, oxanosine, and combinations thereof.
18. The method according to clause 12, wherein the cachexia is associated with illness, trauma, surgery, or burn injury.
19. The method according to clause 18, wherein the illness is selected from the group consisting of cancer, AIDS, HIV, chronic heart failure, and kidney disease.
20. The method according to clause 12, wherein the cachexia is associated with cancer and the method further comprises administering to the subject one or more anti-cancer therapeutics.
21. The method according to any of clauses 12-20, wherein the method further comprises administering to the subject an effective amount of a second active agent selected from the group consisting of propranolol, beta-adrenergic receptor blockers, recombinant human growth hormone, progestin, corticosteroids, metoclopramide, cannabinoids, thalidomide, ghrelin, insulin, nicotinamide mononucleotide, group B vitamins, melatonin, clenbuterol, anabolic steroids, omega 3 fatty acids, non-steroidal anti-inflammatory drugs, (NSAIDs), and combinations thereof.
22. A method for reducing excess bodyweight in a subject in need thereof, the method comprising administering to the subject an effective amount of a PI5P4Kβ agonist.
23. The method according to clause 22, wherein the PI5P4Kβ agonist is selected from the group consisting of hypoxanthine, guanine, guanosine, inosine, guanosine monophosphate (GMP), guanosine diphosphate (GDP), guanosine triphosphate (GTP), inosine triphosphate (ITP), xanthosine triphosphate (XTP), and combinations thereof.
24. The method according to any of clauses 22-23, wherein the subject has as body mass index (BMI) of 25.0 or greater.
25. The method according to any of clauses 22-24, wherein the subject has a BMI of 30.0 or greater.
26. The method according to any of clauses 22-25, wherein the method further comprises administering to the subject an effective amount of a second active agent selected from the group consisting of glucagon, leptin, adrenalin, incretin, nicotinamide mononucleotide, vitamin B group, caffeine, orlistat/vyfat//tetrahydrolipstatin, non-steroidal anti-inflammatory drugs, (NSAIDs), beta-adrenergic receptor antagonists, catabolic steroids, and combinations thereof.
27. A method for increasing the bodyweight of an animal, the method comprising administering to the mammal an effective amount of a phosphatidylinositol 5-phosphate 4-kinase beta (PI5P4Kβ) inhibitor.
28. The method according to clause 27, wherein the animal is selected from the group consisting of cattle, sheep, goats, pigs, rabbits, chickens, ducks, geese, turkeys, horses, fish, frogs, lobster, crab, squid, locust, spiders, and worms.
29. The method according to any of clauses 27-28, wherein the animal is raised for meat consumption.
30. The method according to any of clauses 27-29, wherein the PI5P4Kβinhibitor is selected from the group consisting of 6-thioguanine, I-OMe tyrphostin AG 538, A131, SAR088, NIH-12848, NCT-504, THZ-P1-2, and combinations thereof.
31. The method according to any of clauses 27-30, wherein the PI5P4Kβinhibitor comprises an inosine monophosphate dehydrogenase (IMPDH) inhibitor.
32. The method according to clause 31, wherein the IMPDH inhibitor is selected from the group consisting of mycophenolic acid (MPA), mycophenylate sodium, mycophenylate mofetil, tiazofurin, ribavirin, VX-944, FF-10501, benzamide riboside, mizorbine, 5-ethynyl-1-beta-D-ribofuranosylimidazole-4-carboxamide (EICAR), selenazofurin, thiophenfurin, myricetin, gnidilatimonoein, sappanone A, sanglifehrin, and combinations thereof.
33. The method according to clause 27, wherein the PI5P4Kβ inhibitor comprises a guanosine monophosphate synthetase (GMPS) inhibitor.
34. The method according to clause 33, wherein the GMPS inhibitor is selected from the group consisting of acivicin, angustmycin A, decoyinine, oxanosine, and combinations thereof.
35. The method according to any of clauses 27-35, wherein treating the subject with the PI5P4Kβ inhibitor increases meat quality or yield.
36. A knock-in animal whose genome encodes a mutant PI5P4Kβ kinase, wherein said mutant PI5P4Kβ kinase comprises at least a F205L substitution, wherein the knock-in animal has decreased GTP-sensing activity of the PI5P4Kβ kinase compared to wildtype animals lacking the substitution.

### EXAMPLES

The following examples are given by way of illustration are not intended to limit the scope of the disclosure.

### Example 1. The GTP-Sensing Activity of PI5P4Kbeta is Important for Control of Bodyweight

To assess the functional role of GTP-dependent PI5P4K activity in vivo, F205L knock-in mice were generated by the CRISPR/Cas9 method in C57BL/6J strain and confirmed the on-target mutation. Pip4k2bF^{205L/F205L} mice were born following the Mendelian ratios and showed apparently normal growth with a tendency to increased bodyweight (FIG. 1), as opposed to the decreased adiposity seen in Pip4k2b^{-/-} mice. Primary MEFs from Pip4k2b^{F205L/F205L} mice decreased lysosomal acidification compared to littermate WT primary MEFs (data not shown).

### Example 2. The GTP-Sensing Activity of PI5P4Kbeta is Important for Whole-Body Glucose Metabolism, Organelle Lipid Metabolism, Liver Functions

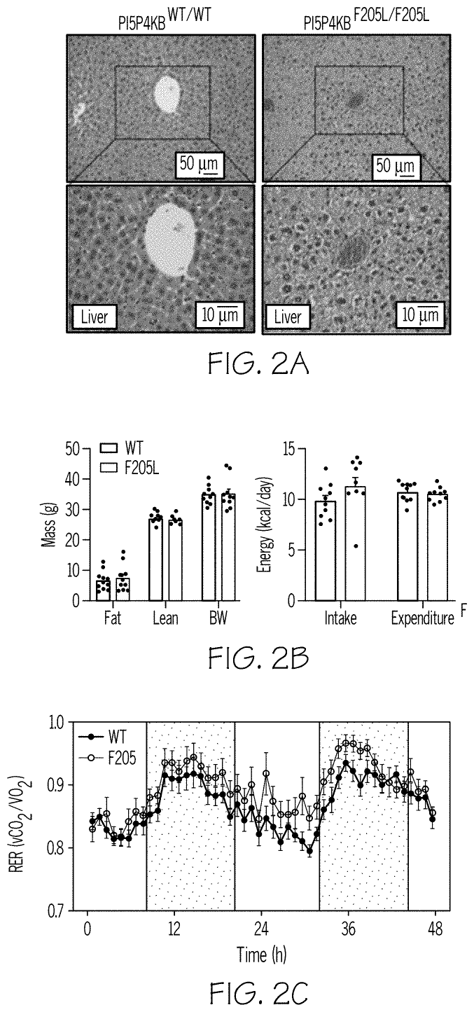

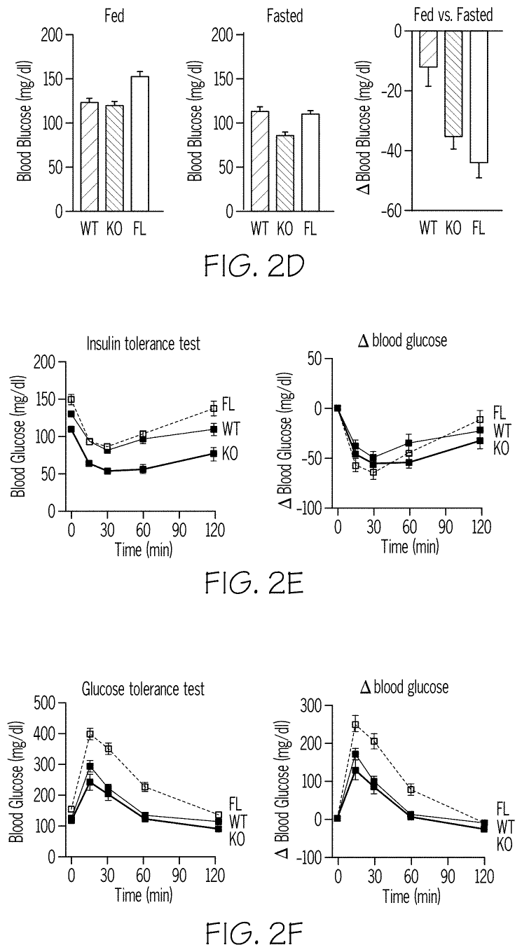

Pip4k2b^{F205L/F205L} and WT mice were subjected to a series of metabolic analyses. Pip4k2b^{F205L/F205L} fed with a standard chow exhibit normal bodyweight with a trend to increase over time (FIG. 2(A)). Among the tested tissues (e.g., brain, muscle, kidney, etc.), the liver showed histological changes, including increased lipid accumulation. Metabolic cage analysis showed that the locomotor activity, food intake, energy expenditure, and body composition of Pip4k2b^{F205L/F205L} mice were comparable to that of WT mice (FIG. 2(B)). Importantly, the respiratory exchange ratios (RER) test suggests that Pip4k2b^{F205L/F205L} mice use less fat and more carbohydrates as the fuel source compared to WT mice (FIG. 2(C)). Pip4k2b^{F205L/F205L} mice exhibited higher blood glucose under fed condition, but interestingly, a larger decrease in blood glucose during the transition from fed to fasting (24 h) state compared to WT mice (FIG. 2(D)), suggesting abnormal regulation of gluconeogenesis. Insulin tolerance test showed that, while the initial response to the insulin challenge is unaffected, Pip4k2b^{F205L/F205L} mice recover the initial glucose baseline faster than WT mice (FIG. 2(E)). Pip4k2b^{F205L/F205L} mice exhibited decreased glucose tolerance (FIG. 2(F)), which is consistent with their higher baseline blood glucose. These results suggest that Pip4k2b^{F205L/F205L} mice alter whole body glucose metabolism and may have enhanced gluconeogenesis.

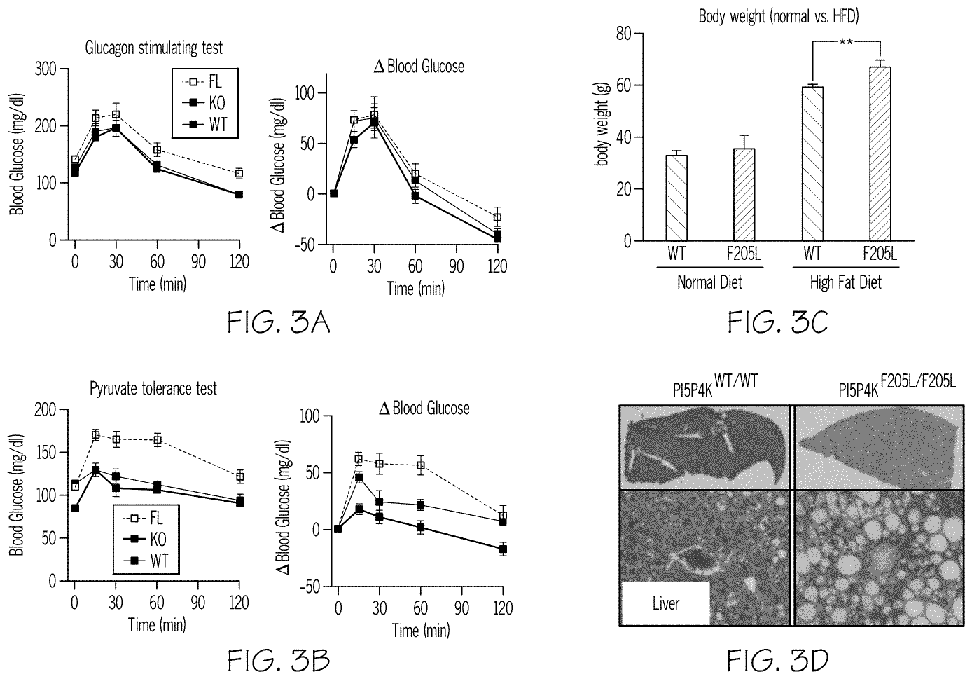

Importantly, the glucagon stimulating test, which induces gluconeogenesis mostly from the stored glycogen, showed no differential responses in Pip4k2b^{F205L/F205L} mice (FIG. 3(A)), suggesting that intracellular components of gluconeogenesis (e.g., PEPCK, G6Pase) of Pip4k2b^{F205L/F205L} liver are likely intact. This notion is further supported by the pyruvate tolerance test, which bypasses the need for fatty acid oxidation and provides fuel for gluconeogenesis itself (FIG. 3(B)). These results suggest that Pip4k2b^{F205L/F205L} alters lipid metabolism, and more particularly, fatty acid oxidation. Strikingly, when fed with a high-fat diet, the bodyweight of Pip4k2b^{F205L/F205L} increased significantly more than that of WT, and Pip4k2b^{F205L/F205L} mice developed more lipid accumulation than WT mice (FIG. 3(C)-(D)). These results indicate that the GTP-sensing activity PI5P4Kβ is important for cellular catabolism (degradation pathway), and thus that controlling this function through modulation of PI5P4Kβ directly or indirectly via changing cellular GTP concentration would provide a mechanism for control of bodyweight, lipid accumulation in the liver and other tissues, and blood glucose. As such, data support that modulation of the GTP-sensing aspect of PI5P4Kβ kinase has application in bodyweight problems in cachexia patients, obese persons, and diabetes and metabolic diseases, as well as increasing meat yield, tenderness, quality, and production of specialty foods (e.g., foie gras, etc.).

### Example 3. Lysosomal Regulation by GTP-Sensor Activity of PI5P4Kbeta

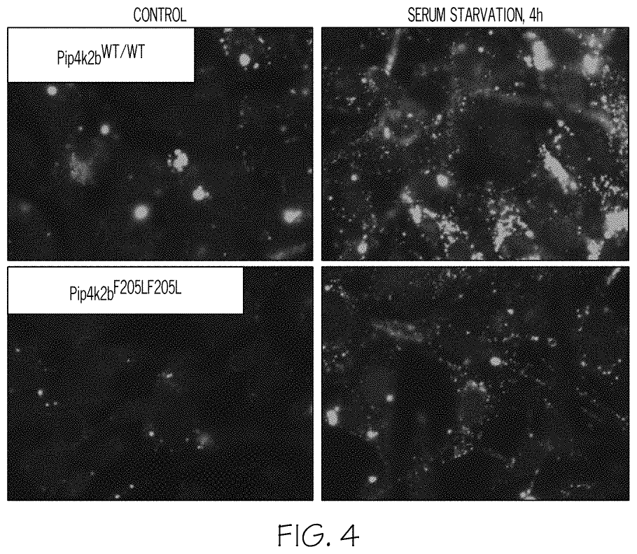

Lysosomes are major cellular degradation stations for all sorts of macromolecules and compose over 60 enzymes for breaking down proteins, polysaccharides, lipids, and nucleotides regenerating their respective building-block molecules (e.g., amino acid, carbohydrate, nucleobase), which are delivered from endocytosis and autophagy. The activity of lysosomes is a key determinant for controlling bodyweight as well as sizes of cells and organelles, and signaling and metabolism, including but not limiting to lipid deposition. Importantly, primary mouse embryonic fibroblasts (MEFs) from Pip4k2b^{F205L/F205L} mouse show decreased lysosomal acidification compared to littermate WT primary MEF (FIG. 4). This suggests that the observed phenotype in GTP-insensitive mice (FIGS. 1-3) are likely due to decreased lysosomal activity, or catabolism, in the GTP-insensitive cells and tissues.

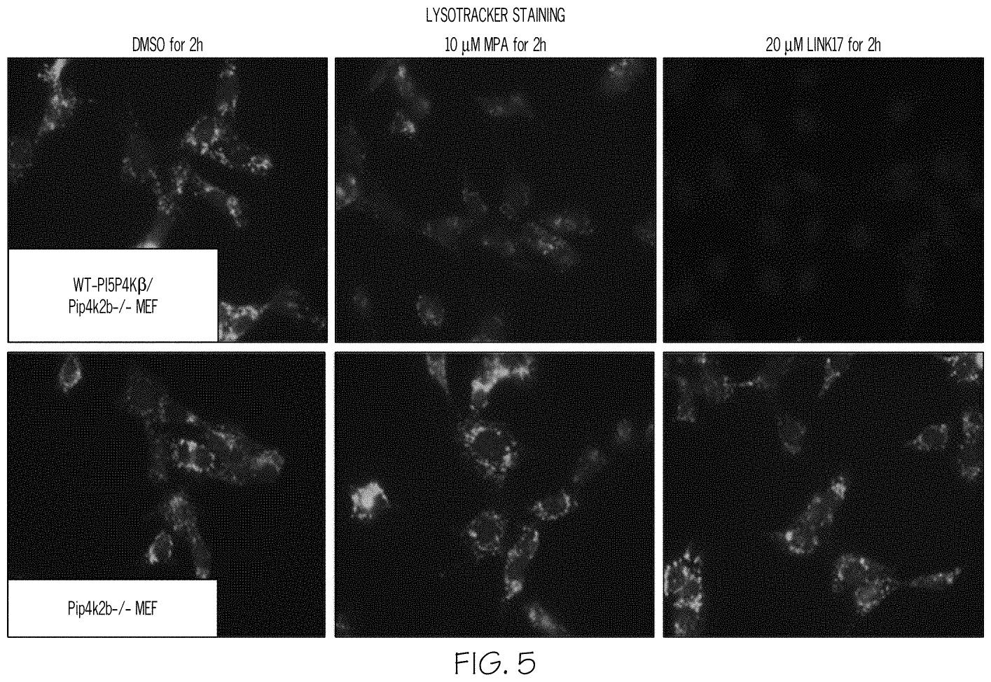

The data further show that pharmacological decrease of cellular GTP levels by mycophenolic acid (MPA) treatment had a deacidification effect in the MEF cells under serum-starved condition (FIG. 5).

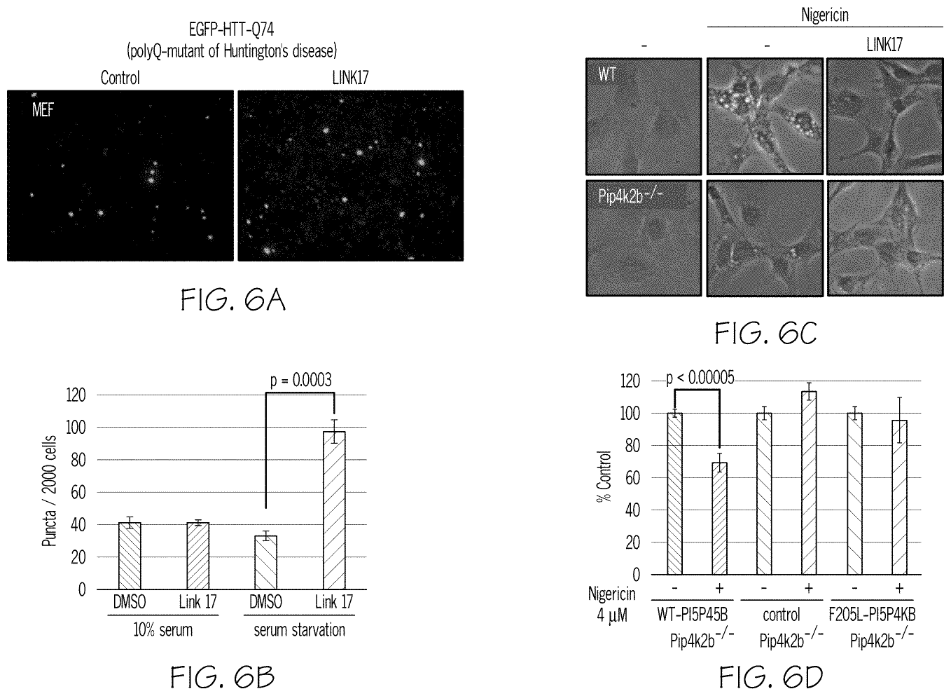

To assess lysosomal-protease activity, mutant Huntington proteins were used, the aggregation forms of which require a lysosome-autophagy activity for clearance. Results showed that PI5P4Kβ inhibitor Link17 treatment significantly increased aggregation of the mutant Huntington protein (FIG. 6(A)-(B)).

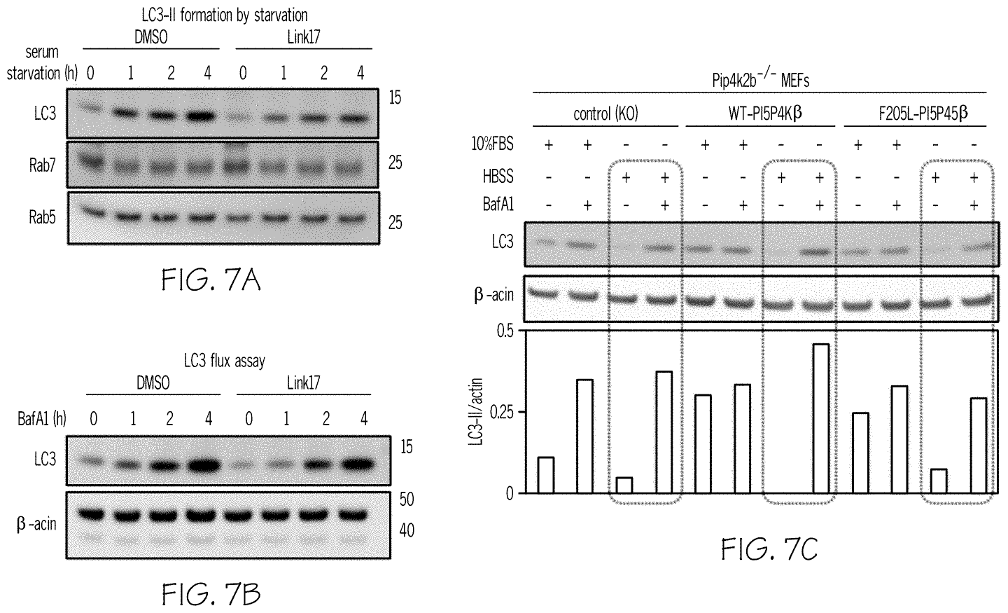

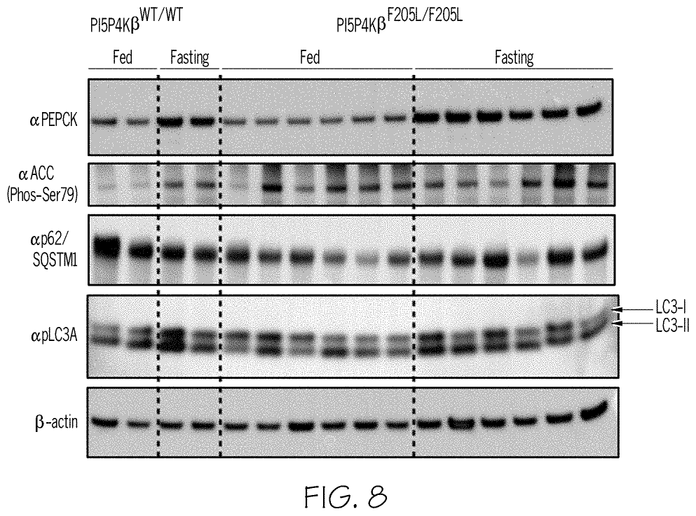

Next, treatment with microbial toxin nigericin, a selective K⁺/H⁺ exchanger that is activated in the lysosome and induces lysosome rupture and following cell death, was assessed. Within 4h after nigericin treatment, hypervacuolization was observed in WT PI5P4Kβ-reconstituted Pip4k2b^{-/-} cells (WT), but not in Pip4k2b^{-/-} cells (FIG. 6(C)). Nigericin treatment decreased cell viability of WT cells, while Pip4k2b^{-/-} and F205L-PI5P4Kβ-reconstituted Pip4k2b^{-/-} (F205L) cells showed tolerance (FIG. 6(D)). The autophagic flux in MEF, 293T, and HCT116 cells was decreased by Link17 treated cells (FIG. 7(A),(B)). In the isogenic MEFs, autophagy was decreased in Pip4k2b^{-/-} and F205L cells, compared to WT cells (FIG. 7(C)). These results indicate that PI5P4Kβ is required for lysosomal acidification and catabolic activities.

Autophagy and lysosomes are activated during fasting and required for β-oxidation of free fatty acids (FFA) that provide the carbon substrate for ketogenesis and mitochondrial bioenergetics (ATP, NADH) to facilitate gluconeogenesis (FIG. 8). These metabolic phenotypes of Pip4k2b^{F205L/F205L} mice resemble the mice suppressing lysosomal lipase and autophagy. Strikingly, Pip4k2b^{F205L/F205L} livers exhibited abnormal ratios of the LC3-I and LC3-II, and aberrant accumulation of p62/SQSTM1, rather than depletion, upon fasting. These results suggest that the GTP-dependent activity of PI5P4Kβ is critical for the autophagy-lysosome system in the liver, which impacts hepatic lipid metabolism and whole-body glucose homeostasis, as a part of the mechanism responsible for the observed phenotypes of the GTP-insensitive mice (FIGS. 1-3).

### Example 4. Regulation of V-ATPase Assembly through Kinase Activity of PI5P4Kbeta.

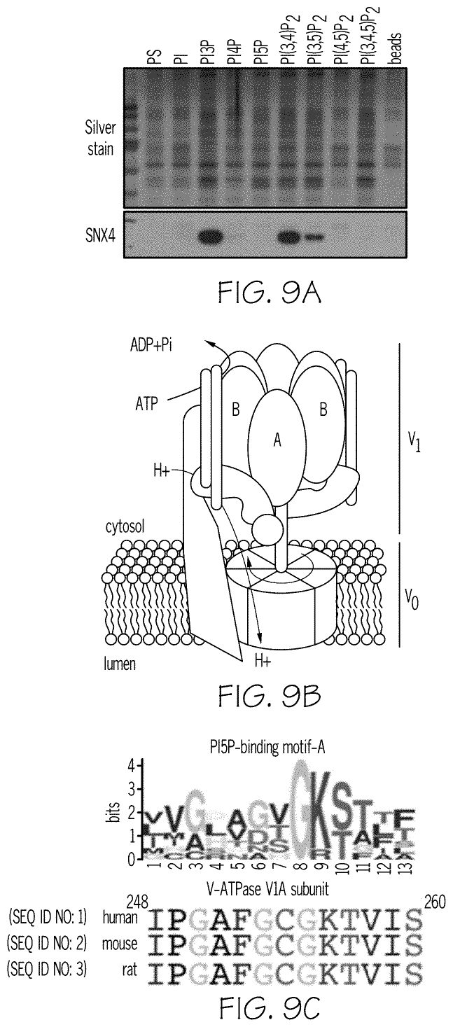

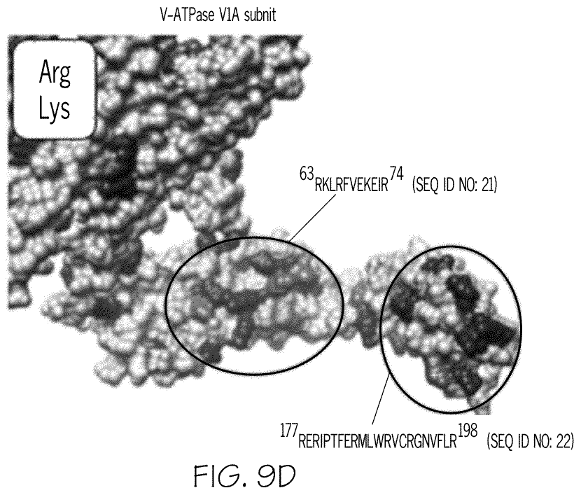

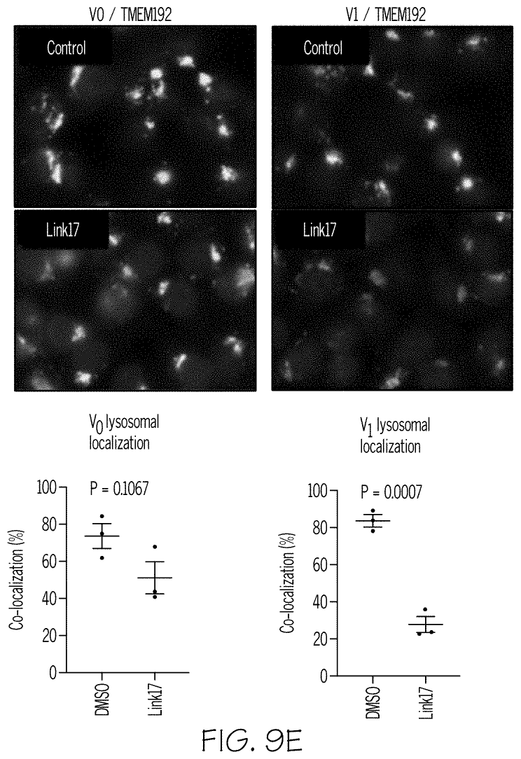

PI5P4Kβ is considered to regulate cell functions through controlling the lipid second messenger PI5P. As of present, no systemic screening for PI5P's effectors has been reported. A proteomic screening was conducted using PolyPIPosomes for the eight species of phosphatidylinositol and pulldown binding proteins from U87MG cell lysates and analyzed by mass spectrometry (FIG. 9(A)). In the PI3P fraction, there are series of previously identified PI3P binders, including SNX4. The validity is extended to the other well-characterized binders for PI(3,4,5)P₃, PI(3,4)P₂, PI(4,5)P₂, and PI4P. Interestingly, in the PI5P fraction, a subunit of V-ATPase, ATPV1A, has been reproducibly identified.

V-ATPase is critical for lysosomal acidification and is composed of a cytosolic V₁ sector that contains sites of ATP hydrolysis and a membrane-bound V₀ sector that performs H⁺ translocation (FIG. 9(B)). The V-ATPase can undergo reversible disassembly for inactivation by phosphorylation and phosphoinositides. In yeast, PI(3,5)P₂ binds to the V₀ segment of V-ATPase and stabilizes V₁-V₀ assembly, while PI4P binds to the V₀ segment to recruit and activate V-ATPase at the Golgi apparatus. Whether mammalian V-ATPase activity is regulated by phosphoinositides remains unknown. Importantly, it was found that the human VIA subunit contains the PI5P-binding motif (FIG. 9(C)). Also, the human V₀-subunit contains the PI5P binding motif, which forms a positively charged pocket (FIG. 9(D)). Strikingly, treatment with Link17 decreased V₁ segment localization to the lysosome (FIG. 9(E)). These results suggest that the V-ATPase assembly is regulated through the kinase activity of PI5P4Kβ.

### Example 5: The ATP Recognition Mode is Shared Among Protein and Lipid Kinases

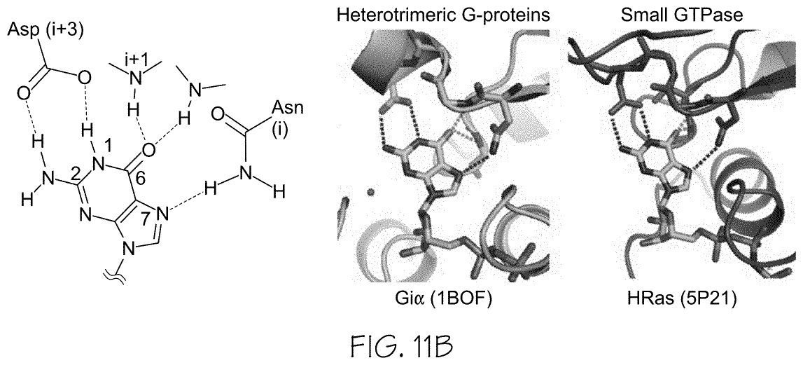

To gain insights into the typical ATP- and GTP-binding modes of proteins and compare them with those of PI5P4Kβ (FIG. 10(B)), 702 unique nucleotide-bound structures were analyzed for protein kinases, phosphoinositide kinases, inositol phosphate kinases (including inositol kinase), as well as 134 G-proteins in the protein database (PDB). The catalytic domains of the kinases consist of two lobes harboring an ATP-binding site at the hinge region. Binding of an adenine base by kinases is characterized by conservation of two mainchain hydrogen bonds to N(1) and N(6) (FIG. 11(A)), while other interactions that are unique in each kinase are also observed. Typically, two hydrogen bonds are formed between the mainchain amide and carbonyl groups from the i+2th and ith residues, respectively. The mode of interaction can be achieved by an extended conformation of the polypeptide and is also conserved in the ATP-binding mode of PI5P4Kβ (FIG. 10(B), bottom). In PI5P4Kβ, the N(1) and N(6) of ATP are recognized by the amide group of Val-204 and carbonyl oxygen of Arg-202, respectively. The N(1) of ATP also forms a hydrogen bond with the Nδ of Asn-203. The cognate ATP kinase PI4P5Kα also interacts with ATP in the same binding mode (FIG. 11(A)).

### Example 6: The Unique GTP-Binding Mode of PI5P4Kβ by the TRNVF Motif

Because the arrangement of a hydrogen donor and acceptor in the guanine base differs from that of the adenine, PI5P4Kβ has a specific GTP-binding mode (FIG. 10(B), top). The mode of interaction is different from that of G-proteins, which utilize the conserved NKXD motif for guanine base recognition (FIG. 11(B)). In G-proteins, the N(1) and NH₂(2) of the guanine base are simultaneously recognized by the sidechain carboxylate of Asp in the NKXD motif In addition, in most cases, the N(7) of the guanine base forms a hydrogen bond with Oδ of Asn in the NKXD motif, and O (6) forms a hydrogen bond(s) with a neighboring i+1th Lys and a remote mainchain amide group(s).

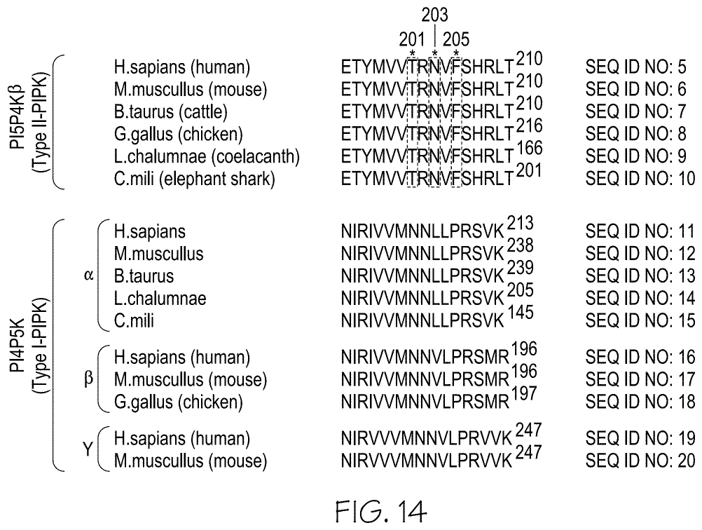

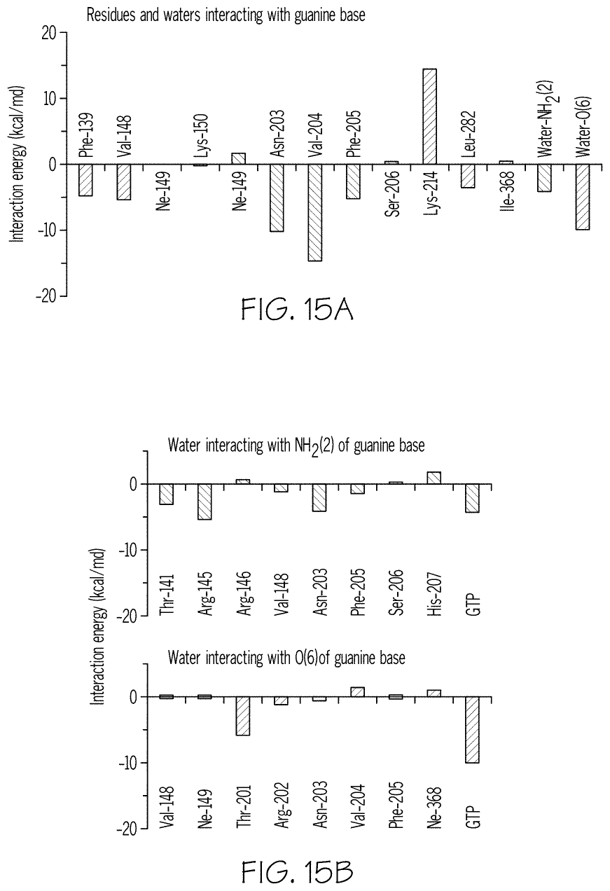

PI5P4Kβ also forms hydrogen bonds to N(1), NH₂(2), and O(6) of the guanine ring; however, the interacting residues are distinct from those of the G-protein. PI5P4Kβ utilizes the TRNVF motif (residues 201-205 in humans) to recognize GTP (FIG. 14). Asn-203 in the TRNVF motif is structurally located at the corresponding position of the conserved Asp residue of the G-protein, as its Oδ and Nδ atoms form direct and indirect hydrogen bonds with N(1) and NH2(2), respectively (FIG. 10(B), top). While G-proteins typically have pico to sub-nano molar affinity to GTP, the affinity of PI5P4Kβ to GTP seems to be much weaker, as its K_{M} value is only ˜100 μM. The indirect hydrogen bond between the Asn-203 Nδ and NH₂(2), which is mediated by a water molecule, might account for the weaker affinity of PI5P4Kβ compared to that of the G-protein. Another characteristic feature of PI5P4Kβ is a hydrogen-bond network around O(6) of the guanine base involving Thr-201, Arg-202, and Val-204 in the TRNVF motif and a water molecule (FIG. 10(B)). These interactions for GTP are enabled by a 1.5 Å shift of the base moiety relative to the ATP. The contribution of the hydrogen-bond network around O(6) for guanine base recognition is also evident from the fragment molecular orbital (FMO) calculation. Both Val-204 and the water molecule held by Thr-201 show an energetically favored interaction to O(6) of guanine base (FIG. 15). Note that the interaction seems to be even stronger than the aforementioned Asn-203 plus water interactions with the NH2(2) position. The shift of the base position promotes a formation of aromatic-aromatic interactions with Phe-205 in the TRNVF motif, which is unique to guanine base recognition. Interestingly, the guanine and adenine base recognition of CKII and PI5P4Kβ has similarity in the hydrogen-bond networks around N(1) and O(6) as well as the 1.5 Å shift of guanine base compared to that of the adenine ring (FIGS. 10(B) and 16); however, CKII uses only mainchain atoms for base recognitions.

The GTP-recognizing TRNVF sequence also serves for the adenine-base recognition and is strictly conserved among PI5P4Kβ proteins (FIG. 14). Therefore, the TRNVF sequence can be designated as a dual nucleotide base-binding motif. Especially, Thr-201, Asn-203, and Phe-205 in the motif would be of importance as their sidechains contribute to the interaction with the guanine base. In contrast, among the ancestral ATP-dependent PI4P5Ks, the MNNψL sequence is conserved, where “ψ” is donated for branched amino acids (FIG. 14). This indicates that PI5P4Kβ has established an atypical mode of GTP recognition, while conserving the canonical ATP-binding mode, by changing a few residues in the MNNwL sequence into the TRNVF motif. Especially, Thr-201 and Phe-205, which establish sidechain interactions with the guanine base, would be of importance due to their unique contributions to the guanine-base recognition.

### Example 7: PI5P4Kβ Can Hydrolyze XTP and ITP

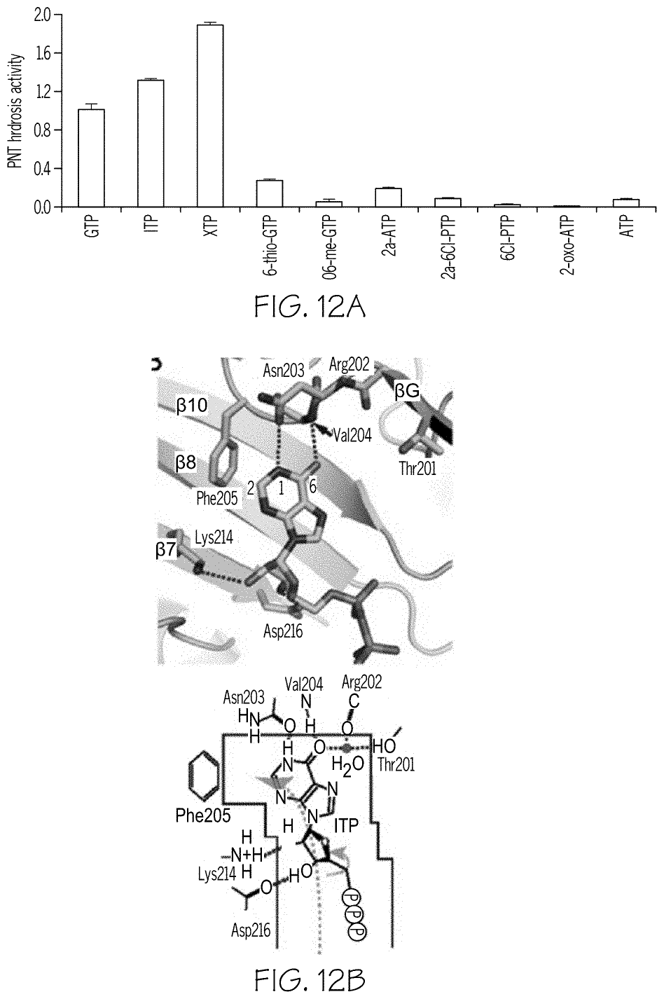

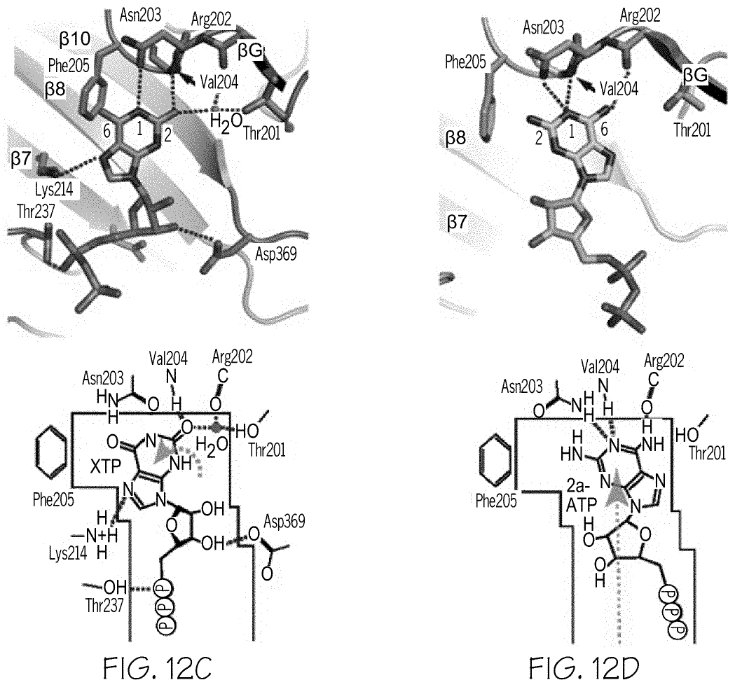

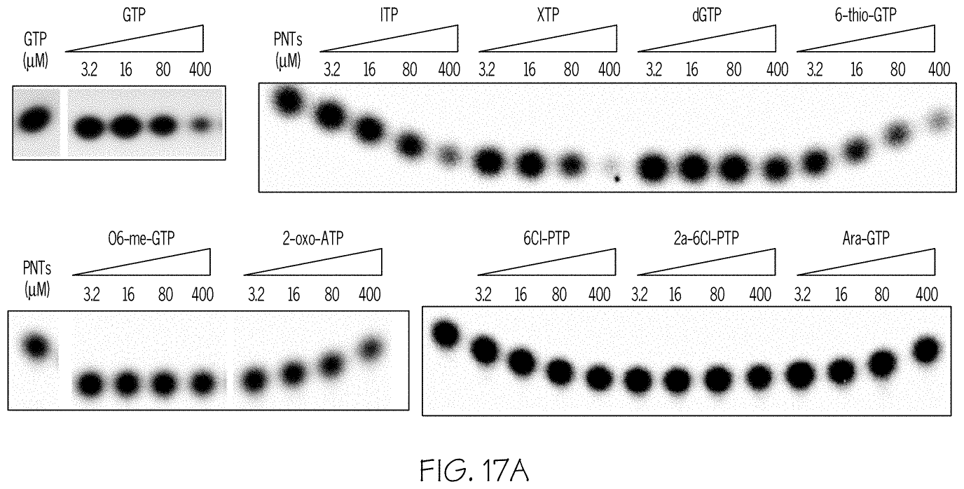

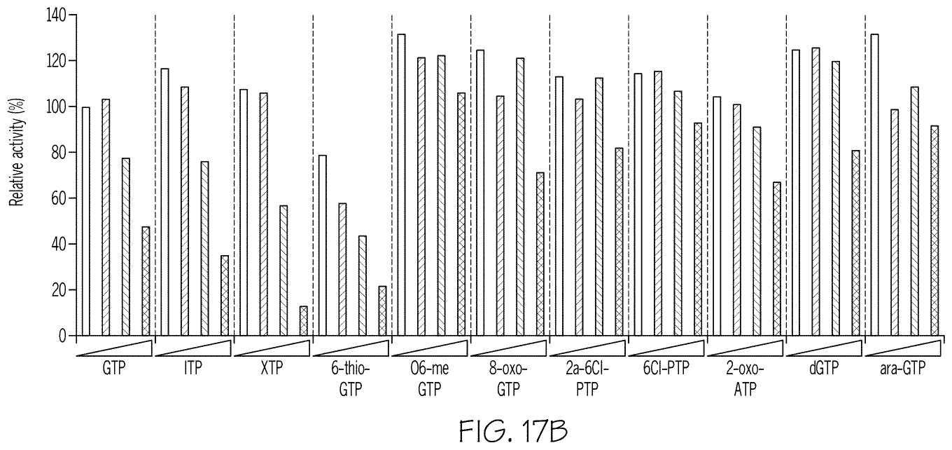

Next, the mechanism of the GTP preference of PI5P4Kβ was investigated using a series of ATP and GTP analogs. Based on analysis of the GTP-PI5P4Kβ interaction (FIG. 10(B)), 10 PNTs with different configurations at the 2nd and 6th positions of the purine base (NH₂(2) and O(6) in guanine base, respectively) were chosen (FIG. 10(A)). The hydrolysis activities of PI5P4Kβ for these PNTs were quantified by an NMR-based assay (data not shown). The intrinsic hydrolysis activity of PI5P4Kβ (i.e., the transfer of phosphoryl to water, instead of PI(5)P) has been shown to reflect the characteristic GTP-preference of the kinase. PI5P4Kβ showed substantial activity with ITP, XTP, 6-Thio-GTP, and 2a-ATP (FIG. 3A), indicating that NH₂(2) is dispensable for the activity of GTP-like PNTs, since both ITP and XTP lack the NH₂(2) moiety. On the other hand, O(6) seems to be required for the activity. ITP and XTP, both of which have the O(6) moiety, showed 1.3- and 1.9-times higher hydrolysis activity compared to GTP, but O6-me-GTP and 2a-6C1-PTP, which lack the O(6) moiety, showed very low hydrolysis activity. In line with this notion, 6-thio-GTP, which possesses sulfate, which is structurally and electrostatically similar to oxygen in the 6th position, can also be utilized by PI5P4Kβ (FIG. 12(A)). A competition assay between these PNTs and GTP showed that the GTP-dependent PI(5)P phosphorylation activity was strongly inhibited by ITP, XTP, and 6-thio-GTP (FIG. 17), supporting the proposition that the specificity of PI5P4Kβ extends beyond GTP due to the strong dependence on the O(6) interaction in the nucleotide recognition. This view is also supported by the larger energetic contribution of the O(6) moiety compared to the NH₂(2) moiety in the FMO interaction analysis between GTP and PI5P4Kβ (FIG. 15).

### Example 8: Crystal Structures of PI5P4Kβ Unveil the Recognition Mechanism of the Active Triphosphorylated Nucleotides

The mechanistic details were analyzed for the extended specificity of PI5P4Kβ beyond GTP using the crystal structures of PI5P4Kβ complexed with any of three PNTs: ITP, XTP, or 2a-ATP. Since the soaking of the 6-thio-GTP broke PI5P4Kβ crystals, the crystal structure of the 6-this-GTP complex could not be obtained. In the 2a-ATP complex, 2a-ATP binds to PI5P4Kβ with a binding mode similar to that of ATP (FIG. 12(D)), except that the N(2) of 2a-ATP and the Phe-205 sidechain seem to form an additional van der Waals interaction. This additional interaction would explain the stronger binding of 2a-ATP compared to ATP. The crystal structure of the ITP complex revealed that the interaction with the inosine base is essentially the same as that with the guanine base (FIG. 12(B)). The water molecule that participates in the hydrogen-bond network around O(6) is less clear in the PI5P4Kf3-ITP complex; however, the presence of the water molecule is evident when the criterion for identifying it is slightly lowered (2σ). Since ITP, which lacks NH₂(2), can reside in the G-site, the contribution of NH₂(2) to the GTP binding to PI5P4Kβ would be minor. Nevertheless, the absence of an interaction with NH₂(2) slightly changes the position of the nucleotide base of ITP relative to GTP, which might explain why the hydrolysis activity of ITP is higher than that of GTP, since the position of the nucleotide base affects the phosphate group positions in the catalytic site.

Surprisingly, XTP has two different but overlapping binding modes in the binding site of PI5P4Kβ. In the first binding mode, XTP is in the G-site forming hydrogen bonds of N(1) and O(6) corresponding to those found in GTP. An indirect hydrogen bond between O(2) and Asn-203 via water was not observed. In the second binding mode, the base of the XTP is flipped by 180° respective to the first binding mode, revealing the distinct XTP-binding mode (FIG. 12(C)). Even after the base flip, XTP forms a hydrogen-bond network similar to the first one; the N(1) occupies an almost identical position within 1 Å difference, and the positions of O(2) and O(6) are merely swapped. As a result, the N(1) still forms a hydrogen bond with Asn-203 Oδ, as observed in the GTP-binding mode. The O(2) of XTP forms bifurcated hydrogen bonds to the mainchain amide group of Val-204 and a water molecule, which in turn forms a hydrogen bond with Oγ of Thr-201. The presence of these two distinct binding modes for XTP would explain the elevated activity of PI5P4Kβ on XTP. Nevertheless, these structural studies showed that ITP and XTP are GTP-type PNTs, in which the hydrogen-bond network around O(6) is critical for the interaction.

### Example 9: Rational PI5P4Kβ Mutants Define the Contribution of Key Residues to the GTP, ATP, and XTP-Binding

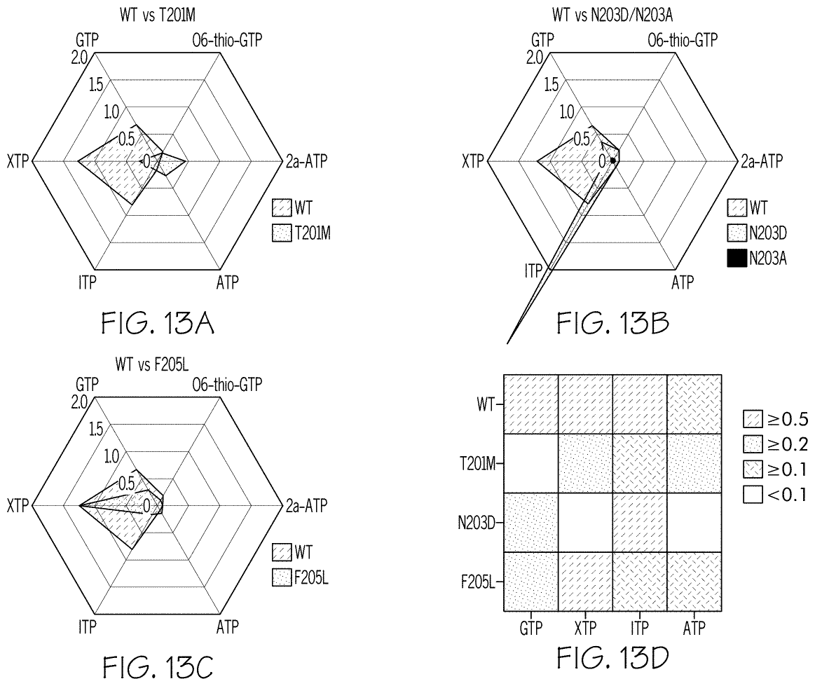

To analyze the contribution of the nucleotide interacting residues to the GTP-, ATP-, and XTP-binding modes, the effect of mutations of Thr-201, Asn-203, and Phe-205 were compared in the TRNVF motif (FIG. 13). Since Thr-201 and Phe-205 are substituted to Met and Leu in PI4P5K (or Type I PIPK) (FIG. 14), respectively, the T201M and F205L mutants could provide insight into the evolutionary change of the base-specificity of PI5P4K.

The functional role of Asn-203 was analyzed, because this is the only invariant residue in the base-recognition loop of PI4P5K and PI5P4K. The mutation of this residue markedly reduced the binding to both ATP and GTP (FIG. 13). The FMO calculation clearly showed the importance of this interaction; approximately ¼ of the interaction energy between the nucleotide base and PI5P4Kβ is contributed by a hydrogen bond between the guanine base and Asn-203 (FIG. 15).

The effects of the T201M (PI5P4KβT201M) and F205L (PI5P4KβF205L) mutations have already been partly reported (Sumita et al., 2016). The decreased GTP-dependent kinase activity of PI5P4KβT201M has been explained by the loss of the hydrogen-bond network around O(6) by the mutation (FIG. 10(B)), and the higher ATP-dependent activity of PI5P4KβT201M seems to have arisen from an additional hydrophobic interaction between the adenine-base and the substituted Met. In the case of PI5P4KβF205L, the loss of the π-π interaction between the guanine base and the aromatic ring of Phe-205 caused the reduction of the GTP-dependent activity. In contrast, the ATP-binding was not affected by the F205L mutation, and PI5P4KβF205L retains an ATP-dependent activity comparable to that of WT PI5P4Kβ.

In addition, the hydrolysis activity of the mutants on four active triphosphorylated nucleotides were analyzed, XTP, ITP, 6-thio-GTP, and 2a-ATP. As expected, PI5P4KβT201M was substantially less active on the GTP-type PNTs (FIGS. 13(A) and (D)), showing the importance of the hydrogen-bond network around O(6). In contrast, PI5P4KβT201M was more active on 2a-ATP and ATP, which shares the ATP-mode interaction (FIG. 10(A)), as these nucleotide bases show additional interactions with the mutated methionine sidechain. Intriguingly, PI5P4KβN203D and PI5P4KβF205L showed much stronger hydrolysis activity for a single NTP other than GTP (FIGS. 13(B) and (C)). PI5P4KβN203D is hyperactive to ITP, while the activities on GTP, XTP, ATP, and 2a-ATP were significantly reduced. In the crystal structure of the PI5P4KβN203D-ITP complex, ITP binds in a similar manner as the WT. Although the crystal structure could not explain the hyper ITPase activity of PI5P4KβN203D, the results suggest the importance of recognizing the 1st position in both GTP- and ATP-mode interactions.

The Phe-205 to Leu mutation makes a protein less active on GTP, ITP, and 6-thio-GTP (FIGS. 4C and D), suggesting the importance of the π-π interaction between the Phe-205 sidechain and the nucleotide bases in the GTP-binding mode (FIG. 15). The diminished susceptibility of XTP might be due to the presence of additional binding modes (the XTP-binding mode), which make the nucleotide less susceptive to the F205L mutation. It should also be noted that the activity on ATP and 2A-ATP were not affected by the F205L mutation, as the sidechain did not contribute to the interaction in the ATP-binding mode.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

The foregoing description is illustrative of particular embodiments of the invention but is not meant to be a limitation upon the practice thereof. While particular embodiments have been illustrated and described, it would be obvious to one skilled in the art that various other changes and modifications can be made without departing from the spirit and scope of the invention. It is therefore intended to cover in the appended claims all such changes and modifications that are within the scope of this invention.

1. A method for treating a metabolic disorder associated with abnormal bodyweight in a subject in need thereof, the method comprising administering to the subject an effective amount of a compound that modulates phosphatidylinositol 5-phosphate 4-kinase beta (PI5P4Kβ) kinase activity,
wherein a PI5P4Kβ inhibitor is administered when the subject suffers from a metabolic disorder associated with an underweight bodyweight; and
wherein a PI5P4Kβ agonist is administered when the subject suffers from a metabolic disorder associated with an overweight or obese bodyweight.

2. The method according to claim 1, wherein the metabolic disorder is selected from the group consisting of cachexia, obesity, type II diabetes, and non-alcoholic fatty liver disease.

3. The method according to claim 2, wherein the metabolic disorder is cachexia and the compound is a PI5P4Kβ inhibitor.

4. The method according to claim 3, wherein the PI5P4Kβ inhibitor is selected from the group consisting of 6-thioguanine, I-OMe tyrphostin AG 538, A131, SAR088, NIH-12848, NCT-504, THZ-P1-2, and combinations thereof.

5. The method of according to claim 3, wherein the PI5P4Kβ inhibitor comprises an inosine monophosphate dehydrogenase (IMPDH) inhibitor.

6. The method according to claim 5, wherein the IMPDH inhibitor is selected from the group consisting of mycophenolic acid (MPA), mycophenylate sodium, mycophenylate mofetil, tiazofurin, ribavirin, VX-944, FF-10501, benzamide riboside, mizorbine, 5-ethynyl-1-beta-D-ribofuranosylimidazole-4-carboxamide (EICAR), selenazofurin, thiophenfurin, myricetin, gnidilatimonoein, sappanone A, sanglifehrin, and combinations thereof.

7. The method according to claim 6, wherein the IMPDH inhibitor is MPA, mycophenylate sodium, mycophenylate mofetil, or combinations thereof.

8. The method according to claim 3, wherein the PI5P4Kβ inhibitor comprises a guanosine monophosphate synthetase (GMPS) inhibitor.

9. The method according to claim 8, wherein the GMPS inhibitor is selected from the group consisting of acivicin, angustmycin A, decoyinine, oxanosine, and combinations thereof.

10. The method according to claim 1, wherein the metabolic disorder is selected from the group consisting of obesity, type II diabetes, and non-alcoholic fatty liver disease and the compound is a PI5P4Kβ agonist.

11. The method according to claim 10, wherein the PI5P4Kβ agonist is selected from the group consisting of hypoxanthine, guanine, guanosine, inosine, guanosine monophosphate (GMP), guanosine diphosphate (GDP), guanosine triphosphate (GTP), inosine triphosphate (ITP), xanthosine triphosphate (XTP), and combinations thereof.

12. A method for treating cachexia in a subject in need thereof, the method comprising administering to the subject an effective amount of a phosphatidylinositol 5-phosphate 4-kinase beta (PI5P4Kβ) inhibitor.

13. The method according to claim 12, wherein the subject is a mammal.

14. The method according to claim 13, wherein the subject is a human.

15. The method according to claim 12, wherein the PI5P4Kβ inhibitor is selected from the group consisting of 6-thioguanine, I-OMe tyrphostin AG 538, A131, SAR088, NIH-12848, NCT-504, THZ-P1-2, inosine monophosphate dehydrogenase (IMPDH) inhibitors, guanosine monophosphate synthetase (GMPS) inhibitors, and combinations thereof.

16. The method according to claim 15, wherein the PI5P4Kβ inhibitor is an IMPDH inhibitor selected from the group consisting of mycophenolic acid (MPA), mycophenylate sodium, mycophenylate mofetil, tiazofurin, ribavirin, VX-944, FF-10501, benzamide riboside, mizorbine, 5-ethynyl-1-beta-D-ribofuranosylimidazole-4-carboxamide (EICAR), selenazofurin, thiophenfurin, myricetin, gnidilatimonoein, sappanone A, sanglifehrin, oxanosine, and combinations thereof.

17. The method according to claim 15, wherein the PI5P4Kβ inhibitor is a GMPS inhibitor selected from the group consisting of acivicin, angustmycin A, decoyinine, oxanosine, and combinations thereof.

18. The method according to claim 12, wherein the cachexia is associated with illness, trauma, surgery, or burn injury.

19. The method according to claim 18, wherein the illness is selected from the group consisting of cancer, AIDS, HIV, chronic heart failure, and kidney disease.

20. The method according to claim 12, wherein the cachexia is associated with cancer and the method further comprises administering to the subject one or more anti-cancer therapeutics.

21. The method according to claim 12, wherein the method further comprises administering to the subject an effective amount of a second active agent selected from the group consisting of propranolol, beta-adrenergic receptor blockers, recombinant human growth hormone, progestin, corticosteroids, metoclopramide, cannabinoids, thalidomide, ghrelin, insulin, nicotinamide mononucleotide, group B vitamins, melatonin, clenbuterol, anabolic steroids, omega 3 fatty acids, non-steroidal anti-inflammatory drugs, (NSAIDs), and combinations thereof.

22-36. (canceled)


SEQUENCE LISTING (23 sequences)

SEQ ID NO: 1 - protein, 13 aa, Homo sapiens
  MISC_FEATURE: V-ATP-ase V1A subunit
IPGAFGCGKTVIS

SEQ ID NO: 2 - protein, 13 aa, Mus musculus
  MISC_FEATURE: V-ATP-ase V1A subunit
IPGAFGCGKTVIS

SEQ ID NO: 3 - protein, 13 aa, Rattus rattus
  MISC_FEATURE: V-ATP-ase V1A subunit
IPGAFGCGKTVIS

SEQ ID NO: 4 - protein, 5 aa, Homo sapiens
  MISC_FEATURE: Short nucleotide base-recognition motif
TRNVF

SEQ ID NO: 5 - protein, 16 aa, Homo sapiens
ETYMVVTRNVFSHRLT

SEQ ID NO: 6 - protein, 16 aa, Mus musculus
ETYMVVTRNVFSHRLT

SEQ ID NO: 7 - protein, 16 aa, Bos taurus
ETYMVVTRNVFSHRLT

SEQ ID NO: 8 - protein, 16 aa, Gallus gallus
ETYMVVTRNVFSHRLT

SEQ ID NO: 9 - protein, 16 aa, Latimeria chalumnae
ETYMVVTRNVFSHRLT

SEQ ID NO: 10 - protein, 16 aa, Callorhynchus milii
ETYMVVTRNVFSHRLT

SEQ ID NO: 11 - protein, 16 aa, Homo sapiens
NIRIVVMNNLLPRSVK

SEQ ID NO: 12 - protein, 16 aa, Mus musculus
NIRIVVMNNLLPRSVK

SEQ ID NO: 13 - protein, 16 aa, Bos taurus
NIRIVVMNNLLPRSVK

SEQ ID NO: 14 - protein, 16 aa, Latimeria chalumnae
NIRIVVMNNLLPRSVK

SEQ ID NO: 15 - protein, 16 aa, Callorhynchus milii
NIRIVVMNNLLPRAIK

SEQ ID NO: 16 - protein, 16 aa, Homo sapiens
NIRIVVMNNVLPRSMR

SEQ ID NO: 17 - protein, 16 aa, Mus musculus
NIRIVVMNNVLPRAMR

SEQ ID NO: 18 - protein, 16 aa, Gallus gallus
NIRIVVMNNVLPRALK

SEQ ID NO: 19 - protein, 16 aa, Homo sapiens
NIRVVVMNNILPRVVK

SEQ ID NO: 20 - protein, 16 aa, Mus musculus
NIRVVVMNNVLPRVVK

SEQ ID NO: 21 - protein, 11 aa, Homo sapiens
RKLRFVEKEIR

SEQ ID NO: 22 - protein, 22 aa, Homo sapiens
RERIPTFERMLWRVCRGNVFLR

SEQ ID NO: 23 - protein, 416 aa, Homo sapiens
  MISC_FEATURE: Human PI5P4Kbeta protein sequence
MSSNCTSTTAVAVAPLSASKTKTKKKHFVCQKVKLFRASEPILSVLMWGVNHTINELSNVPVPVMLMPDDFKAYSKIKVDNHLFNKENLPSRFKFKEYCPMVFRNLRERFGIDDQDYQNSVTRSAPINSDSQGRCGTRFLTTYDRRFVIKTVSSEDVAEMHNILKKYHQFIVECHGNTLLPQFLGMYRLTVDGVETYMVVTRNVFSHRLTVHRKYDLKGSTVAREASDKEKAKDLPTFKDNDFLNEGQKLHVGEESKKNFLEKLKRDVEFLAQLKIMDYSLLVGIHDVDRAEQEEMEVEERAEDEECENDGVGGNLLCSYGTPPDSPGNLLSFPRFFGPGEFDPSVDVYAMKSHESSPKKEVYFMAIIDILTPYDTKKKAAHAAKTVKHGAGAEISTVNPEQYSKRFNEFMSNILT